(19) United States
(12) Patent Application Publication
(10) Pub. No.: US 2025/0166723 A1
(43) Pub. Date: May 22, 2025

(54) MACHINE LEARNING APPLICATIONS TO PREDICT BIOLOGICAL OUTCOMES AND ELUCIDATE UNDERLYING BIOLOGICAL MECHANISMS

(71) Applicant: Fred Hutchison Cancer Center, Seattle, WA (US)

(72) Inventors: Taranjit Gujral, Seattle, WA (US); Siddharth Vijay, Redmond, WA (US)

(73) Assignee: Fred Hutchison Cancer Center, Seattle, WA (US)

(21) Appl. No.: 18/841,319

(22) PCT Filed: Feb. 24, 2023
(86) PCT No.: PCT/US2023/063290

§ 371 Date: Aug. 23, 2024

Related U.S. Application Data
(60) Provisional application No. 63/314,306, filed on Feb. 25, 2022.

(51) Int. Cl. G16B 5/00; G16B 15/30; G16B 25/10; G16B 40/20; G16H 50/50

(52) U.S. Cl. CPC G16B 5/00; G16B 15/30; G16B 25/10; G16B 40/20; G16H 50/50

(57) ABSTRACT

Systems and methods for modeling highly complex biological relations in machine-learned models, such as neural networks (e.g., such as deep neural networks (DNNs)), to predict biological outcomes and elucidate underlying mechanisms are described. The systems and methods utilize recursive feature elimination and scoring and can be utilized to prioritize particular compounds or treatments for clinical development and direct new avenues of research and development based on elucidated mechanisms.

### CROSS-REFERENCE TO RELATED APPLICATION

This application is a U.S. National Phase Patent Application based on International Patent Application No. PCT/US2023/063290 filed Feb. 24, 2023, which claims priority to U.S. Provisional Patent Application No. 63/314,306 filed Feb. 25, 2022, the entire contents of each of which are incorporated by reference herein.

### FIELD OF THE DISCLOSURE

The current disclosure provides systems and methods for modeling highly complex biological relations in machine-learned models, such as neural networks (e.g., such as deep neural networks (DNNs)) to predict biological outcomes and elucidate underlying mechanisms. The systems and methods can be utilized to prioritize particular compounds or treatments for clinical development and direct new avenues of research and development based on elucidated mechanisms.

### BACKGROUND OF THE DISCLOSURE

Advances in high-throughput drug profiling and large-scale molecular-omics data collection, coupled with exponentially improving computational power, have opened avenues for applying artificial intelligence (AI)-driven methods to identify candidate ‘hit’ molecules in physiology and biological sciences, which is regulated through complex, multifaceted signaling networks within and between cells. Previous attempts to accurately model these relations results in “black box” models. That is, while previously-used methods may accurately predict an outcome, they do not sufficiently advance scientific knowledge because the basis for the outputs remain unknown.

### SUMMARY OF THE DISCLOSURE

The current disclosure provides systems and methods for predicting biological responses to certain inputs. For example, the systems and methods can be used to predict efficacy of a potential treatment in an individual patient or a patient population. The systems and methods also elucidate underlying biological mechanisms of the biological response (e.g., treatment success or failure). The systems and methods include training a machine learning model to model a response of a biological system (e.g., a molecule, protein, cell, tissue system, organ system, organism) to an input (e.g., a molecule, compound, protein, or the like). The model can perform recursive feature elimination and scoring of inputs.

### BRIEF DESCRIPTION OF THE FIGURES

Some of the drawings submitted herein may be better understood in color. Applicant considers the color versions of the drawings as part of the original submission and reserves the right to present color images of the drawings in later proceedings.

FIGS. 1A-1E. Application of the disclosed framework for interpretable neural network (e.g., DNN) modeling of kinase inhibitor responses. (1A) Schematic of the disclosed modeling framework. The modeling framework may include developing preliminary neural network(s) that may model a response of a biological system to training inputs applied to the biological system. Next, recursive feature elimination is applied to rank the training inputs based on contribution to the response of the biological system and to deduce the optimal number of variables used to build the final network (sometimes referred to herein as the DeepKinX network, at least as regards a first example regarding human protein kinase identification). Finally, the disclosed network is used to predict synergistic drug combinations and to select kinases for subsequent experimental validation. (1B) A plot showing the LOOCV MSE of DeepKinX-Mes and DeepKinX-Epi after each round of elimination. The number of kinases in the round with the lowest MSE is labeled for each model. (1C) A plot showing a comparison between viability predicted by DeepKinX-Epi and DeepKinX-Mes and observed viability of Huh7 WT cells and Huh7-Fzd2 cells in response to 43 kinase inhibitors and DMSO control. The Mean squared error (MSE), Mean absolute error (MAE), and Pearson Correlation are listed. (1D) A bar plot showing the top ‘selective’ kinases predicted by the disclosed modeling framework based on selectivity score (rank order difference). All kinases above a 150 selectivity score (epithelial RKI rank—mesenchymal RKI rank) are plotted. (1E) A heatmap showing p-value of correlation between the expression (mRNA) of the indicated kinases and mesenchymal-type tumor state from 17 different cancers. Only those with a significant correlation have marked p-values.

FIGS. 2A-2C. Optimization. (2A) A heatmap showing the LOOCV MSE of networks built with selected combinations of batch sizes and epochs. Yellow regions indicate low relative errors. (2B). Heatmaps showing the LOOCV MSE of networks built with selected combinations of optimizers and weight initializations. The underlying activation function (ReLU, ELU, SELU) used to build each set of networks is indicated in each of the 3 heatmaps. (2C). Heatmaps showing the LOOCV MSE of networks built with selected combinations of hidden layer quantities and nodes per hidden layers. The underlying dropout rate (0 or 0.25) used to build each set of networks is indicated in both heatmaps.

FIGS. 3A-3C. Recursive Kinase Elimination. (3A) A bar plot showing the top 16 predicted ‘important’ kinases by DeepKinX-Mes in mesenchymal cells (Huh7+Fzd2) and their relative importance score based on MSE increase after permutation. (3B). A bar plot showing the top 22 predicted ‘important’ kinases by DeepKinX-Epi in epithelial cells (Huh7 WT) and their relative importance score based on MSE increase after permutation. (3C). A plot showing a comparison between DeepKinX-Epi-predicted viability and observed viability of Huh7 WT cells in response to 43 kinase inhibitors and DMSO control. The MSE, MAE and Pearson Correlation are listed.

FIGS. 4A, 4B Experimental validation of DeepKinX identified mesenchymal cell-specific kinases (4A). Quantitative, real-time PCR results for E-Cadherin (CDH1) expression in Huh7-Fzd2 cells transfected with transient siRNA knockdowns targeting various kinases. Presented as the fold change compared to a non-targeting siRNA. (4B) Quantitative changes in cell migration as assessed by a wound-healing assay in Huh7-Fzd2 transfected with transient siRNA targeting indicated kinases. siFZD2 is included as positive controls. Data are presented as mean±SEM of at least three biological replicates. * denote p<0.05.

FIG. 5. DeepKinX-predicted mesenchymal cancer cell-specific kinases are upregulated in mesenchymal cancer cells. Plot shows the relative abundance of the DeepKinX-predicted ‘selective’ kinases in Huh7 WT versus Huh7+Fzd2 cells measured by mass spectrometry. The kinases are ordered by largest upregulation in abundance in Huh7+Fzd2 as compared to Huh7 WT.

FIGS. 6A-6D. DeepKinX models predict effective drug combinations. (6A) A heatmap showing the predicted effect of pairwise drug combinations of all 428 drugs using DeepKinX-Mes. Drugs are ordered by predicted efficacy of single drugs. (6B) A plot showing relative viability of Huh7-Fzd2 cells treated with indicated inhibitors tested at 500 nM or pairwise combinations tested at 250 nM each. Bars represent mean of five independent replicates. Error bars represent SEM. ****p<0.0001, one-way ANOVA with two-tailed Holm-Sidak multiple comparisons test. (6C) A plot showing relative viability of Huh7-Fzd2 cells treated with indicated inhibitors tested at 500 nM or three-drug cocktails at 165 nM each. Bars represent mean of three independent replicates. Error bars represent SEM. *p<0.05, **p<0.01, ***p<0.001, ****p<0.0001, one-way ANOVA with two-tailed Holm-Sidak multiple comparisons test. (6D) Heatmaps showing the experimentally-determined relative kinase inhibition of the top 30 kinases predicted by DeepKinX-Mes and experimentally validated by the three most synergistic drug combinations (two pairwise drug combos and one three-drug combo). The kinases are ranked by the last ‘combination’ column in each heatmap, which is the linear combination of the inhibition values of the combo drugs.

FIG. 7. Pairwise kinase inhibitor combination and kinase activity profiles. Five heatmaps showing the individual and combined inhibition of the 50 most ‘important’ DeepKinX-predicted kinases by 5 different sets of pairwise drug combinations. The combo column displays a linear combination of the two drugs' individual observed inhibition.

FIG. 8. Cocktail of three kinase inhibitor combination and kinase activity profiles. Six heatmaps showing the individual and combined inhibition of the 50 most ‘important’ DeepKinX-predicted kinases by 6 different sets of three-drug cocktails. The combo column displays a linear combination of the three drugs' individual observed inhibition.

FIGS. 9A-9D. Modeling single-cell RNA seq data from melanoma patients for immunotherapy response. (9A) The UMAP (Uniform Manifold Approximation and Projection) and bar plot (9B) showing the immune cell distribution between non-responders and responders of the checkpoint immunotherapy. (9C) The performance of SVM (support vector machine)-based model using all cell types (left), macrophages only (middle), and CD8 T cell (right) in predicting immunotherapy response. (9D) The performance of XGBoost-based models in predicting melanoma patients' immune response.

FIGS. 10A-10E. DeepGeneX identifies genesets that can predict patient response to immunotherapy. (10A) A schematic illustrating DeepGeneX framework. (10B) A plot showing the LOOCV accuracy of DeepGeneX after each round of feature elimination. The number of genes used to build the model in each round is also indicated. (10C) The importance score of the top 6 genes predicted by DeepGeneX. (10D) A plot showing the importance score of the top 6 genes predicted by DeepGeneX in each round of recursive gene elimination. (10E) A confusion matrix showing the accuracy of DeepGeneX-based predictions of immunotherapy response in 19 patients.

FIGS. 11A-11C. Optimization of DeepGeneX models. (11A) A heatmap showing the LOOCV binary cross-entropy of networks built with selected combinations of batch sizes and epochs. Red regions indicate high accuracy. (11B) A heatmap showing the LOOCV binary cross-entropy of networks built with selected combinations of optimizer and weight initializers. (11C) A heatmap showing the LOOCV binary cross-entropy of networks built with selected combinations of the number of hidden layers and nodes per hidden layer.

FIGS. 12A, 12B. Expression and distribution of DeepGeneX-predicted marker genes in responders and non-responders population. (12A) Violin plots comparing the overall expression distribution across all immune cells between responders and non-responders. (12B) The UMAPs comparing the difference in the expression of the six marker genes: SELL, CCR7, GZMB, GZMH, LGALS1, and WARS in different immune cell populations. ***denote p<0.0005, Mann Whitney U test.

FIGS. 13A-13C. Validation of DeepGeneX-identified marker genes in other cancers. (13A) Violin plots showing the difference in expression of six marker genes between responders and non-responders in patients with basal cell carcinoma. *denotes p<0.05, **denote p<0.005, *** denote p<0.0005, Mann Whitney U test. (13B) Log-rank test results comparing the overall survival difference between patients with the favorable expression pattern of marker genes (high SELL/CCR7 and low LGALS1/WARS) and patients with unfavorable expression patterns across all cancer types in the TCGA database. (13C) Kaplan-Meier survival curve comparing patients with favorable/unfavorable marker genes' expression pattern within TCGA-SKCM (melanoma) dataset, p-val<0.005, log-rank test.

FIGS. 14A-14C. Pathway enrichment and cell-cell interactions of MOLW-high macrophages. (14A) GO pathways enriched in Motw-high from non-responders compared to macrophages from responders' population. NES; normalized enrichment score (14B) A dot plot showing the expression and distribution of ligands predominantly secreted by macrophages (in red) that could contribute to the CD8 T cell difference between responders and non-responders. (14C) A heatmap showing the potential targeted genes in CD8 T cells in response to ligands expressed in non-responders' macrophages. Ligands with bold italic font are differentially expressed in MΦ^{LW}-high only.

FIGS. 15A, 15B. Expression of ligands in macrophages and targeted genes in CD8 T cells in responder and non responders. (15A) A heatmap showing the expression of ligands that could impact CD8 T cells under three conditions: (1) Mf^{LW}-high from non-responders (2) Mf^{LW}-low from non-responders and (3) Mf from responders. CD80, CD86, TNFSF10, TNFSF 13B and ICAM1 are upregulated uniquely in Mf^{LW}-high. CXCL2, VEGFA, CCL20, CXCL11, HBGEF and IL1B are overexpressed in both Mf^{LW}-high and Mf^{LW}-low compared to responders' macrophages (15B) Violin plot showing the differential expression of targeted genes in CD8 T cell between responders and non-responders. non-responders have overexpression of GAPDH, EZH2, VCAM1, PRF1,TSCCD3 (GILZ), STAT1, FKBP5, IFIT3, CTNNB1 and BCL2L11; responders expressed more in BTG2, CD44, FOS, MALAT1 and NR4A2.

### DETAILED DESCRIPTION

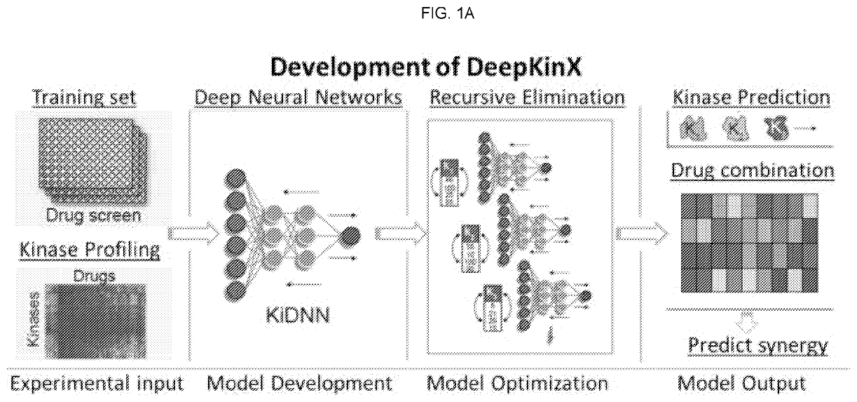

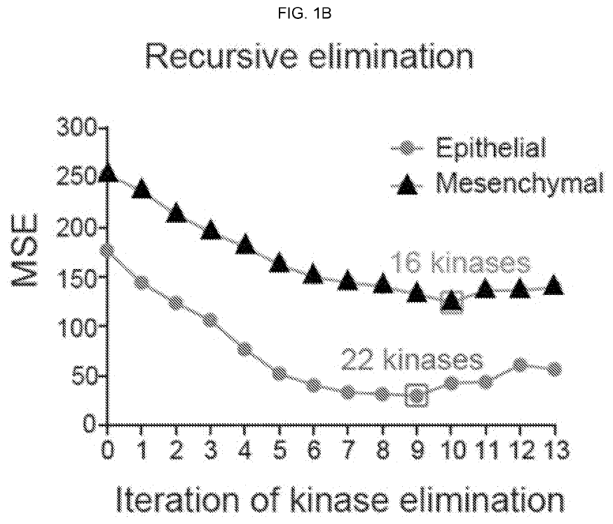

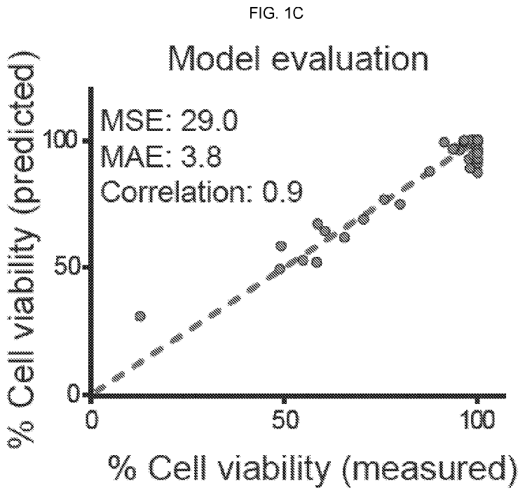

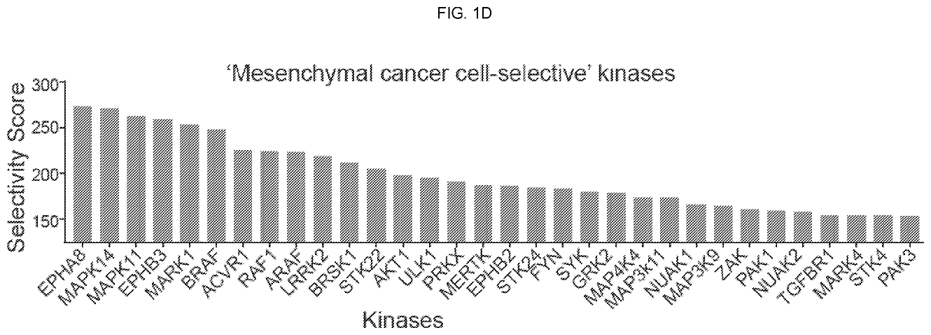

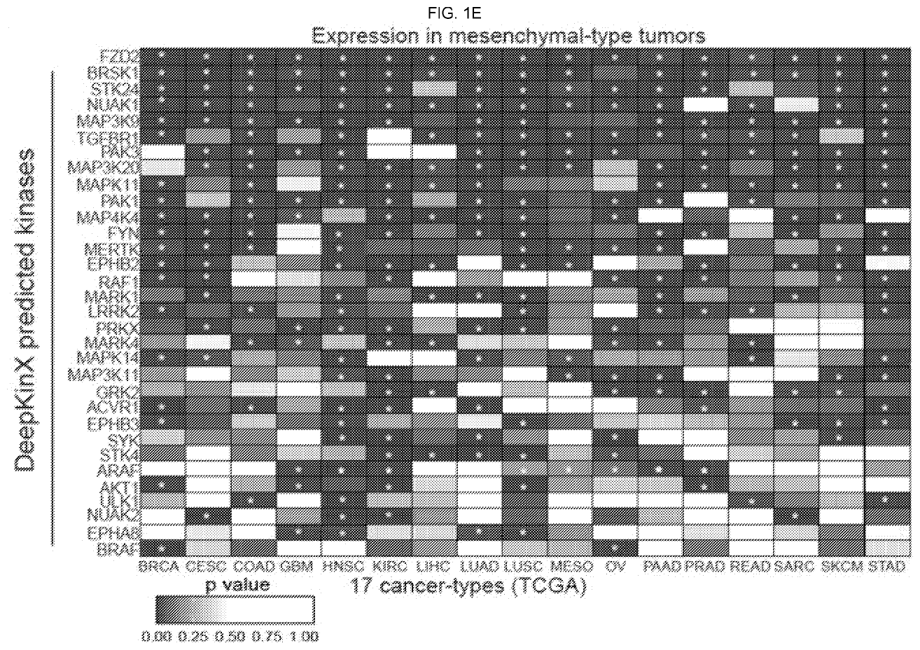

To address the limitation of current prediction models, the current disclosure provides a machine-learning model that may comprise, for example, a neural network-based modeling framework, sometimes referred to herein as a deep neural network, “DNN Model” (FIG. 1), although it is understood that additional or alternate neural network types could be used. For example, a DNN refers, generally to a depth of a neural network and not, necessarily, the components thereof. It is understood that the techniques discussed herein may use a feed-forward neural network, artificial neural network (ANN), recurrent neural network (RNN), convolutional neural network (CNN), radial basis function neural network, multilayer perceptron (MLP), and/or the like. In a first example, the techniques discussed herein may be applied to a DNN. The neural network and recursive feature elimination techniques discussed herein may integrate and accurately model complex biological response data to predict biological outcomes (of, for example, a potential treatment) while also elucidating underlying biological mechanisms underlying the predictions. Current neural networks provide no interpretability or target deconvolution for why they generate outputs. The neural network and recursive feature elimination discussed herein elucidate underlying mechanisms of the neural network that would otherwise be uninterpretable and can be used to uncover previously-unknown biological phenomena, driving the state of biological knowledge forward into new areas of research and development. Additional or alternate machine-learned models may be used to determine the predictions discussed herein. For example, such machine-learned models may additionally or alternatively include a transformer, support vector machine, or other self-attention model; tree-based model(s) such as a random forest or other decision tree based model (which may involve generating hundreds or thousands of trees); and/or the like.

The techniques (e.g., model(s) and/or process(es)) discussed herein generally relate to a neural network trained to receive a set of inputs (e.g., candidate compounds to treat or diagnose a condition, activity profiles associated with a type of input). Examples of inputs include kinase inhibitors, cytokine data, biomarkers, immune checkpoint blockers (ICB), RNA sequence(s), DNA sequences, cells, immune cells, antibiotics, vaccine candidates, etc. In some examples, the neural network may be trained to predict a response of a biological system to the inputs. For example, according to the DeepGeneX example discussed further herein, the input data may comprise training data that comprises multiple RNA sequences for a group of subjects and lab data indicating a response of a these subjects to a therapy or treatment, such as pharmaceutical treatment. For example, for a particular subject, an RNA sequence may be determined using a biological sample received from the subject and lab data identifying a biological response to treatment may be determined during or after a course of treatment administered to the subject.

The biological system could include, for example, a protein, cell, tissue system, organ system, organism, or the like. A training data set for training such a neural network may include training inputs (e.g., proteins, biomarkers, immune checkpoint blockers (ICBs)) for which a response of the biological system has been quantified. In some examples, to determine the training inputs themselves, a feature set associated with each training input may be determined for each training input. For example, the feature set may include data about a molecule or an activity profile associated with the input. To give kinase inhibitors as an example, a feature set for a kinase inhibitor may include a quantitative inhibition profile determined in association with that kinase inhibitor, and the training inputs may collectively include all the quantitative inhibition profiles for all the kinase inhibitors that are being explored.

The neural network may be trained by providing iteratively: providing a training input to the neural network; determining, by the neural network, a predicted response of the biological system to the training input; determining a difference (e.g., quantified as an error) between the predicted response and a quantified/observed response (e.g., part of the training data) of the biological system to the training input; and altering parameter(s) (e.g., weight(s) and/or bias(es)) of one or more components of the neural network to reduce the difference/error. This process may be iteratively repeated in some examples, until a convergence of the error is reached, a number of iterations has been reached, and/or the error is less than a threshold error. In some examples, once this initial training has been completed or during the training, a baseline error of the model may be determined. This baseline error may indicate how well the neural network performs for the training inputs, for which the biological response is already known. This training may enable the neural network to determine a prediction that an input induces a change in one or more cells of a biological sample, for example, such as a molecular response to a drug inhibitor.

To give a tangible example of such training, the training set of inputs may include features of a group of kinase inhibitors and the biological response may be a cellular response, such as cell viability or transition of the cell to an epithelial or mesenchymal state. Quantification of cellular response to the kinase inhibitors could include, for example, determining a score (e.g., an average score) associated with a cellular response to exposure of a cell or cell line to a particular kinase inhibitor. For example, such a score may indicate a progression towards an epithelial or mesenchymal state, as an example of one type of cellular response that could be quantified. Once scores have been generated for each kinase inhibitor, training the neural network may start with hyperparameter tuning, which may include a grid search or other optimization technique that tests and/or optimizes hyperparameters of different preliminary neural networks to reduce the baseline error of the respective preliminary neural networks. Hyperparameters may include, for example, a type of activation function (e.g., sigmoid, linear, rectified linear unit (ReLU), Gaussian error linear unit (GELU), exponential linear unit (ELU), or the like), a number of hidden layers of the model, pooling layer placements and/or types, whether/how much (e.g., percentage, ratio)/how frequently (e.g., every n number of layers, where n is a positive integer) dropouts or skip layers are used and their placement in the neural network, a number of hidden layers in the neural network, a number of nodes in a layer, and/or the like.

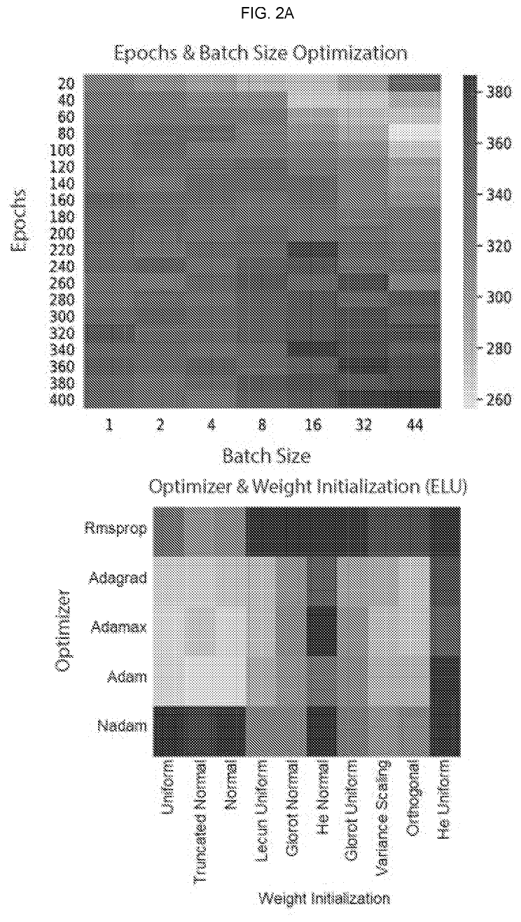

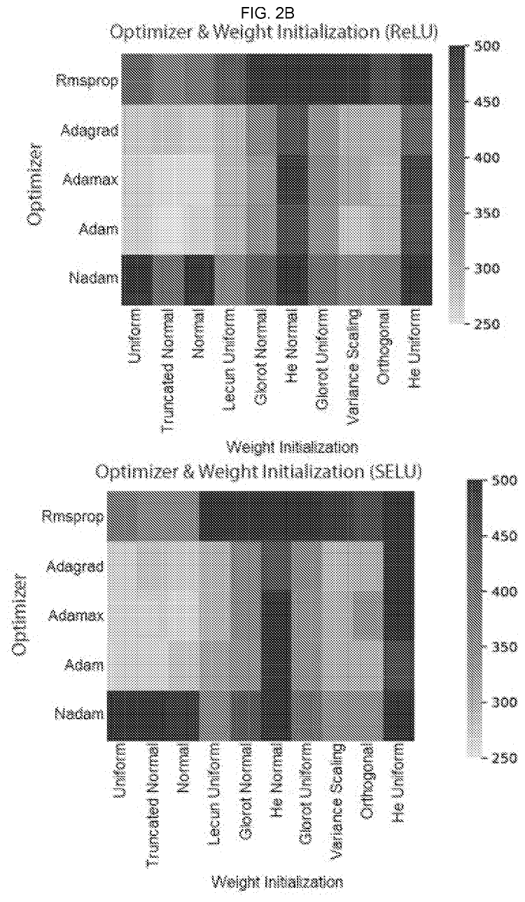

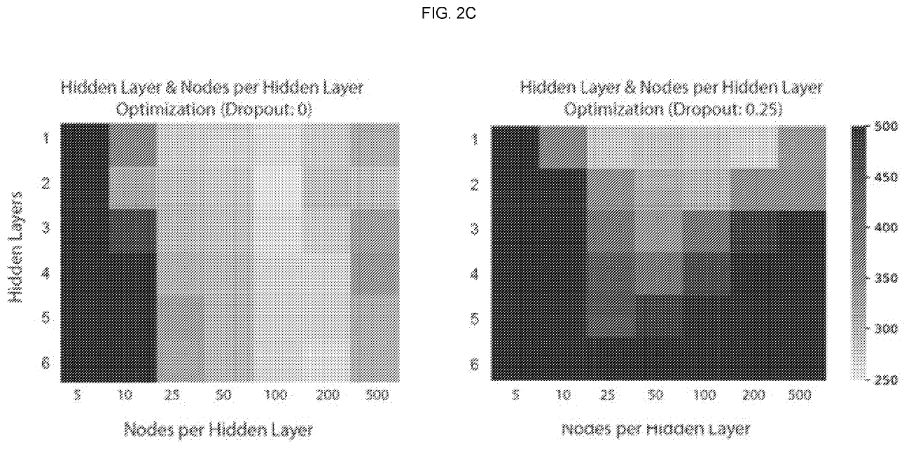

In some examples, the hyperparameters may further include training parameters that may affect how the training occurs, such as a batch size of the training data, a number of epochs (e.g., number of cycles of neural network tuning based on the loss function/gradient decent) completed, the type of loss optimization used (e.g., which gradient descent function is used) and parameters related thereto that control the learning rate of the optimization algorithm or a specific optimization algorithm type (e.g., Adam, Rmsprop, Adagrad, Adamax, Nadam), what type of loss may be determined by the optimization algorithm (e.g., least absolute deviations (L1 loss), least square error (L2 loss), mean squared error (MSE), binary cross entropy, least squares optimization, ridge loss, ridge optimization, or the like), weight initialization technique (e.g., un iform, truncated, normal, Lecun uniform), etc. In some examples, the hyperparameters may be chosen based at least in part on any of the errors above, e.g., L2 or MSE, and/or by conducting leave-one-out cross-validation (LOOCV) or k-fold cross validation (k-fold CV) as a preliminary model is trained and tested. Either cross-validation technique may avoid overfitting and may determine a performance metric associated with a preliminary model as part of the process. Regardless of how the neural network is regularized, the process may include determining a set of hyperparameters to use based at least in part on performance metrics of the preliminary neural networks, such as by determining a set of neural network hyperparameters that is associated with a performance metric that indicates that the set of neural network hyperparameters outperformed other sets of neural network hyperparameters (e.g., the performance metric indicates a minimum error or an error that is less than errors associated with the other hyperparameters). These hyperparameters may define the structural attributes of the neural network, which may then be trained using the training inputs and quantified biological responses, such that the resultant neural network may predict a response of a biological system to a particular input. The figures contained herein illustrate an example hyperparameter optimization at FIGS. 2A-2C.

However, merely being able to predict the response to an input provides no target deconvolution or, put simply, any knowledge of the molecular mechanisms that cause the biological response. Knowing what molecular and/or biological mechanisms are acted upon/triggered by an input allows development of targeted therapies, such as pharmaceutical or other therapies. Once the neural network has been trained using the training inputs and quantified biological responses related thereto, recursive feature elimination may be used to identify underlying molecular mechanisms that give rise to the observed responses.

In a first example, the neural network may have a number of input nodes equal to the number of training inputs provided. To determine the molecular mechanisms at work, the techniques may include determining a test set of inputs that remove one or more of the training inputs. In other words, if the training set of inputs includes 50,000 kinases (or kinase activity profiles), the test set of inputs may include 49,999 kinases (or kinase activity profiles). Kinase activity profiles are but one example of the sort of data that may be used as input data for the machine-learned technique discussed herein. In yet another example, the training inputs may include activity profiles for gene activity in contributing to a biological response. For example, this may include 26,000 genes and the ICB responses thereto. In an additional or alternate example, the training set of inputs may comprise RNA sequencing data for a subject and a measured biological response of the subject to a treatment or therapy. This measured biological response (e.g., the activity profile) may include one or more features that quantify a biological reaction of the subject and/or the subject's cells or other biological matter to the administration of a treatment or therapy to the subject.

As discussed further herein, the ML model discussed herein may use input biological data, such as an RNA sequence associated with a subject, to predict a biological response. A difference between this predicted biological response and the measured biological response determined for that subject may be used to determine a baseline error associated with the ML model, which may be used to determine significant molecular mechanisms, as discussed in more detail herein. Regardless of what the input data is, generally speaking the input data may indicate the presence, absence, or other characteristic (e.g., beta-value, ratio, confidence interval, count) of a feature of a biological sample associated with a subject. For example, one feature (from among a plurality of features) indicated by an RNA sequence generated from a biological sample received from a subject may indicate whether or not a specific gene is expressed, such as by fragments per kilobase per million mapped fragments (FPKM), reads per kilobase of transcript per million mapped reads (RPKM), transcripts per kilobase million (TPM), or the like. In some examples, multiple features may be associated with a single gene. It is understood that the input data may be highly dependent on the type of input data. For example, a feature of kinase activity may quantify kinase activity, as opposed to fragment counts, which may be RNA sequence-specific.

Regardless, according to the techniques discussed herein, an ML model may be trained (as discussed in more detail above) to receive input data quantifying features of a biological sample received from a subject and to use the input data to predict a biological response of the subject to a particular treatment or therapy. A difference between the predicted biological response and a measured biological response may be used to determine the baseline error discussed above. For example, this baseline error may be determined based at least in part on a pre-defined cost function, such as the mean squared error, binary cross entropy, or some other error or a cost function that applies further functions to the error. In some examples, the baseline error may be determined per feature of the input data and may be averaged across multiple samples received from different subjects for that same feature. For example, two examples of such a function may be given by:

$$C ( M S E ) = \frac{1}{m} {\sum }_{i = 1}^{m} {( {y}_{i} - \hat{y} )}^{2} \qquad Equation 1 ( a )$$

$$or$$

$$C ( Binary Cross Entropy ) = - \frac{1}{m} {\sum }_{i = 1}^{m} {y}_{i} log {\hat{y}}_{i} + ( 1 - {y}_{i} ) log ( 1 - {\hat{y}}_{i} ) \qquad Equation 1 ( b )$$

where m is the number of biological responses (in an instance were multiple biological responses have been quantified), y is the observed biological response, and ŷ is the predicted biological response by the neural network and where the baseline error may be given by:

$${e}_{baseline} = \frac{1}{m} {\sum }_{i = 1}^{m} C ( f ( {y}_{i} , {X}_{i} ) ) \qquad Equation 2$$

where the neural network is defined by f and the training set, including both the observed biological response, y, and training inputs, X, are defined by [y, X], and where X=x₁ . . . x_{q}, where q is the number of input features.

Returning to the RNA sequencing example, the input data may comprise RNA sequencing data for m samples received from m number of subjects. A specific RNA sequence associated with an i-th individual may indicate multiple features associated with a gene and the RNA sequence may further comprise multiple genes, each of which may be indicate one or more features. The ML model discussed herein may determine a predicted biological response using the specific RNA sequence associated with the i-th individual and a baseline error may be determine for each feature of each gene (or to simplify, at least one feature of one of the genes sequenced). The baseline errors determined for a particular feature across multiple samples may be averaged to determine an average baseline error associated with a particular feature.

In some examples, the baseline error may be used in conjunction with a permutation error to determine one or more features that most strongly affect the biological response being predicted by the ML model. This increases the interpretability of the ML model by uncovering the inner workings of the ML model's training, which isn't human interpretable, to expose those features that are being most heavily relied upon by the ML model in predicting a biological response. Once a baseline error (or average baseline error) has been determined for each feature of input data according to the discussion above, the input data may be permuted, as permuted input data, and re-provided to the ML model. The ML model may determine, based at least in part on the permuted input data, an updated output that indicates a new predicted biological response. A permutation error may be determined by determining a difference between the new predicted biological response and a measured biological response. A difference between the baseline error and the permutation error may be determined. The larger this difference, the more significantly the feature of the input data affects the predicted biological response, as discussed further below.

In some examples, the training input data may be permuted or test input data (e.g., training data reserved for use after the ML model is trained to a sufficient degree of accuracy) may be permuted. The result of permuting the input data may be called permuted data, permuted inputs, or altered inputs herein. The permutation may include altering a feature itself. For example, where the input data includes activity profiles for a group of kinases, a particular feature of an activity profile may be modified (e.g., a value associated with a discrete portion of the activity profile, which may identify a particular molecular activity, may be altered, such as by increasing or decreasing the value by a set amount or clamping the value to a maximum or minimum value associated with the activity). In an additional or alternate example, features from different samples may be shuffled. For example, in an RNA sequencing example, the RNA sequences for m samples may each indicate different values associated with a particular gene, e.g., an RNA sequence of a first sample received from a first subject may indicate a first value associated with the particular gene and an RNA sequence of a second sample received from a second subject may indicate a second value associated with the particular gene. Permuting the input data by shuffling may include swapping the first value and the second value while holding the rest of the values indicated by the respective RNA sequences constant. In some examples, the shuffling may be randomized (e.g., which sample value is swapped with another sample value).

In an additional or alternate example, the process may further comprise determining a Spearman correlation between features and/or using a clustering algorithm, such as k-means, hierarchical clustering, or the like, to identify correlated and/or similar features. In such an example, two or more features having a Spearman's rank correlation that meets or exceeds a threshold correlation or feature determined to be within a same cluster may be permuted at the same time. For example, normally during permutation for an RNA sequence feature, only one feature is permuted, such as by shuffling two or more values of two or more different samples associated with a particular gene while holding the rest of the values associated with the rest of the genes constant. According to this additional or alternate example, values for two or more genes of a same cluster or having a correlation coefficient that meets or exceeds a threshold can have their values permuted at the same time while holding the rest of the genes constant. In other words, for each gene of a subset identified by Spearman correlation and/or cluster, the values may be shuffled or otherwise permuted, while values associated with the remaining genes outside the subset may be held constant.

This additional or alternate example helps to minimize the effect of bias in training the ML model that may be introduced by highly correlated input data. For example, if a first feature is highly correlated with a second feature, permuting values of the input data associated with the feature won't overly result in an increased importance score since the ML model may rely more heavily on the second feature, whose values are being held constant, resulting in a permutation error that may be similar to the baseline error. In the additional or alternate examples, the input data associated with both the first feature and the second feature may be permuted, resulting in an increased importance score since the correlated features are being permuted, which should result in an increased permutation error since the ML model's bias has been mitigated by permuting input data associated with both of the correlated features.

After altering the input data to achieve a set of permuted features, the permuted features may be provided as input to the neural network trained as described above and the neural network may determine a predicted response of the biological system to the permuted features. The predicted response determined for the permuted input may be used to determine an error (i.e., a permutation error) associated with the permutation by determining a difference between the predicted output and the observed/quantified biological response identified in the training data. This process may be repeated hundreds, thousands, tens of thousands, or more times for each feature of the input data. For example, for an RNA sequence, 10,000 iterations of shuffling may be determined for a first gene, resulting in 10,000 permutation errors. These permutation errors may be averaged and the average permutation error may be associated with the first gene. This process may then be repeated for a second gene and a second average permutation error may be determined in association with the second gene. Accordingly, the process discussed herein may comprise tens or hundreds of millions of iterations of shuffling to determine an average permutation error in association with each gene indicated by an RNA sequence. Any other number of iterations may be used, such as 100 iterations 500 iterations, 5,000 iterations, or a 100,000 iterations, to give but a few examples.

An importance score may be associated with the feature that was altered based at least in part on the permutation error and the baseline error. For example, the importance score associated with the altered feature may be based on the relative error that the permutation caused. Put simply, the importance score associated with a particular input feature may quantify whether or how badly exclusion or modification that feature affects accuracy of the trained neural network. In some examples, the importance score may be a difference between the baseline error and the permutation error. Once a permutation error has been determined, the relative importance (RI) of the feature that was altered may be determined according to:

$$RI = {e}_{permutation} - {e}_{baseline} \qquad Equation 4$$

In some examples, the relative importance may be a score (i.e., an importance score) quantifying a reliance of the observed biological response on the input feature. For example, a lower relative importance may indicate a lower relationship of the input feature to causing the observed biological response, whereas a higher score may indicate a greater reliance of the biological response on the input feature.

In some examples, the input features may be ranked according to importance score. Based at least in part on the ranking, the model may then determine a subset of inputs/input features. The subset of inputs may be determined by including a top r percentage of the input features according to importance score ranking in the subset of inputs or by excluding a bottom s percentage of inputs according to importance score ranking, where r and s are different positive integers. For example, in one example, the top 50% of input features may be retained as the subset or, in another example, a bottom 25% of the input features may be excluded to form the subset.

In some examples, the process described above may be repeated until a completion event is reached. For example, the subset of input data may become the new input data and the process of permuting the input data (i.e., the subset determined by the last iteration), determining importance scores associated with the input data, and determining a subset of input data may be repeated until a completion event is reached. The completion event may be reaching 100% accuracy by the model, which may indicate that the input features of the last iteration identify the molecular mechanism that causes the biological response that was observed. In an additional or alternate event, the completion event may additionally or alternatively include meeting or exceeding an accuracy threshold, determining a subset of input features having a number of input features that is equal to or less than a threshold number of input features, or the like.

In some examples, the multiple models may produce multiple errors to predict a molecular response. For example, the model may be a first model and the error may be a first error, and the subset may be a first subset. Based at least in part on the first model, a second model may be determined, which may determine a second error. Based at least in part on the second error, the second model may then re-rank the target compound within the set of target compounds. The second model may then, based at least in part on re-ranking the target compounds, determine a second subset of target compounds.

Overall, this disclosure presents a machine-learned approach that uses recursive feature elimination and significance scoring to reduce a complex dataset into a clinically actionable dataset with high accuracy, such as 90%+, 95%+, 98%+, 99%+ accuracy. This disclosure also demonstrates that the neural network accurately models complex biological data and elucidates the underlying molecular mechanisms behind the predictions. The neural network is a generally applicable approach that can predict the effects from any dataset and in any disease context, given a training set of measurements. As such, the neural network is a significant step towards a more robust machine-based strategy for predicting phenotypic and clinical response to therapeutics with a complex mechanism of action, and as such, an essential addition to the current set of methodologies in this area.

Biological Inputs. In some examples, inputs are complex biological inputs, for example, an RNA-sequencing (RNA-Seq) dataset. RNA-Seq is often used to identify, analyze, and quantify the expression of a particular gene at a moment in time and under experimental conditions. RNA-Seq can utilize one or more next generation sequencing platforms, allowing rapid analysis of various sized genomes compared to previous sequencing technologies. Typically, RNA-Seq consists of some or all of identifying a biological sample of interest that has been subjected to one or more experimental conditions, isolating RNA therefrom, obtaining RNA reads, aligning the RNA reads to a transcriptome (e.g., of a transcriptome library), and performing various downstream analyses, such as differential expression analysis.

In some examples, inputs include a spatial transcriptomics dataset. Spatial transcriptomics is a technology used to spatially resolve RNA-sequence data, including mRNAs, present in individual tissue sections. Spatially barcoded reverse transcription primers are applied in an ordered fashion to a surface (e.g., the surface of a microscope slide referred to as a gene expression assay slide), thus enabling the encoding and maintenance of positional information throughout the RNA sample processing and sequencing. When a fresh-frozen tissue section is attached to the surface, the spatially barcoded primers bind and capture RNAs from the adjacent tissue. Post RNA capture, reverse transcription of the RNA occurs, and the resulting cDNA library incorporates the spatial barcode and preserves spatial information. The barcoded cDNA library enables data for each RNA transcript to be mapped back to its point of origin in the tissue section.

In some examples, complex biological inputs include a single-cell RNA sequencing (scRNA-Seq) process. Single-cell RNA-sequencing, (scRNA-seq) partitions RNA-Seq data into libraries with unique DNA barcodes for each RNA sample cell of origin. scRNA-Seq, as this enables profiling the transcriptomes of many cells in parallel. A typical scRNA-Seq experiment can profile millions of cells. The release of the first million-cell dataset occurred in 2017.

In some examples, complex biological inputs include epigenetic measures. Epigenetic alterations in DNA provides valuable prognostic information. Epigenetics refers to changes in gene expression that are not due to mutations (i.e. changes in the sequence, such as loss or gain of nucleotides, of a gene). Thus, epigenetics is a reversible regulation of gene expression caused by several mechanisms other than mutation.

The most widely studied epigenetic modification is DNA methylation. Other epigenetic changes include changes to the three-dimensional structure of DNA, histone protein modification, micro-RNA inhibitory activity, imprinting, X-inactivation, and long-distance chromosomal interaction.

Deep mutational scanning libraries of proteins can also be used as inputs. In particular embodiments, a deep mutational scanning library includes protein variants with 19 possible amino acid substitutions at each amino acid position and all possible codons of the associated 63 codons at each amino acid position. In particular embodiments, a deep mutational scanning library includes variants with every possible codon substitution at every amino acid position in a gene of interest with one codon substitution per library member. In particular embodiments, a deep mutational scanning library includes variants with one, two, or three nucleotide changes for each codon at every amino acid position in a gene of interest with one codon substitution per library member. In particular embodiments, a deep mutational scanning library includes variants with one, two, or three nucleotide changes for each codon at two amino acid positions, at three amino acid positions, at four amino acid positions, at five amino acid positions, at six amino acid positions, at seven amino acid positions, at eight amino acid positions, at nine amino acid positions, at ten amino acid positions, etc., up to at all amino acid positions, in a gene of interest with one codon substitution per library member. In particular embodiments, the start codon is not mutagenized. In particular embodiments, the start codon is Met.

In particular embodiments, a deep mutational scanning library includes variants with one, two, or three nucleotide changes for each codon at every amino acid position in a gene of interest with more than one codon substitution, more than two codon substitutions, more than three codon substitutions, more than four codon substitutions, or more than five codon substitutions, per library member. In particular embodiments, a deep mutational scanning library includes variants with one, two, or three nucleotide changes for each codon at every amino acid position in a gene of interest with up to all codon substitutions per library member. In particular embodiments, 20% of library members can be wildtype, 35% can be single mutants, and 45% can be multiple mutants. Multiple mutants can be advantageous, and the sequencing required by the systems and methods disclosed herein is so efficient that using 20% of reads on wildtype is not a problem. Additionally, there are alternative (more complex) mutagenesis methods that give a larger proportion of single amino acid mutants [see, e.g., Kitzman, et al. (2015) Nature Methods 12:203-206; Firnberg & Ostermeier (2012) PLoS One 7: e52031; Jain & Varadarajan (2014) Analytical Biochemistry 449:90-98; and Wrenbeck, et al. (2016) Nature Methods 13:928].

In particular embodiments, a deep mutational scanning library includes or encodes all possible amino acids at all positions of a protein, and each variant protein is encoded by more than one variant nucleotide sequence. In particular embodiments, a deep mutational scanning library includes or encodes all possible amino acids at all positions of a protein, and each variant protein is encoded by one nucleotide sequence. In particular embodiments, a deep mutational scanning library includes or encodes all possible amino acids at less than all positions of a protein, for example at 10%, 20%, 30%, 40%, 50%, 60%, 70%, 80%, 90%, 95%, 96%, 97%, 98% or 99% of positions. In particular embodiments, a deep mutational scanning library includes or encodes less than all possible amino acids (for example 10%, 20%, 30%, 40%, 50%, 60%, 70%, 80%, 90%, 95%, 96%, 97%, 98% or 99% of potential amino acids) at all positions of a protein. In particular embodiments, a deep mutational scanning library includes or encodes less than all possible amino acids (for example 10%, 20%, 30%, 40%, 50%, 60%, 70%, 80%, 90%, 95%, 96%, 97%, 98% or 99% of potential amino acids) at less than all positions of a protein, for example at 10%, 20%, 30%, 40%, 50%, 60%, 70%, 80%, 90%, 95%, 96%, 97%, 98% or 99% of positions. In particular embodiments, a deep mutational scanning library including a set of variant nucleotide sequences can collectively encode protein variants including at least a particular number of amino acid substitutions at at least a particular percentage of amino acid positions. “Collectively encode” takes into account all amino acid substitutions at all amino acid positions encoded by all the variant nucleotide sequences in total in a deep mutational scanning library.

In particular embodiments, a codon-mutant library can be generated by PCR, primer-based mutagenesis, as described in US2016/0145603. In particular embodiments, a codon-mutant library can be synthetically constructed by and obtained from a synthetic DNA company such as Twist Bioscience (San Francisco, CA). In particular embodiments, methods to generate a codon-mutant library include: nicking mutagenesis as described in Wrenbeck et al. (2016) Nature Methods 13:928-930 and Wrenbeck et al. (2016) Protocol Exchange doi:10.1038/protex.2016.061; PFunkel (Firnberg & Ostermeier (2012) PLoS ONE 7(12): e52031); massively parallel single-amino-acid mutagenesis using microarray-programmed r oligonucleotides (Kitzman et al. (2015) Nature Methods 12:203-206); and saturation editing of genomic regions with CRISPR-Cas9 (Findlay et al. (2014) Nature 513(7516): 120-123).

(ii) Alternative Models. While the current embodiment relies on the use of a neural network, any type of machine learning model may be used. A machine learning model, according to example embodiments of the present disclosure, may be a defined computation algorithm executable by one or more processors of a computing system to perform tasks that include processing input having various parameters and outputting results. A machine learning model may include, for example, a layered model such as a deep neural network, which may have a fully-connected structure, may have a feedforward structure such as a convolutional neural network (“CNN”), may have a backpropagation structure such as a recurrent neural network (“RNN”), or may have other architectures suited to the computation of particular tasks. Tasks may include, for example, classification (e.g., responder/non-responder to a therapy), matching, regression, and the like. Tasks may provide output for the performance of functions supporting the prediction and modeling of molecular mechanisms.

A machine learning model may run on a computing system, which includes computing resources which may run a machine learning model to perform one or more tasks as described above. In some examples, machine learning models may be pre-trained with parameters, and may be stored on storage of the computing system and, upon execution, loaded into memory of the computing system.

The Examples below are included to demonstrate particular, non-limiting embodiments of the disclosure. Those of ordinary skill in the art will recognize in light of the present disclosure that many changes can be made to the specific embodiments disclosed herein and still obtain a like or similar result without departing from the spirit and scope of the disclosure.

### Experimental Examples. Example 1. DeepKinX-Interpretable Deep Learning Framework for Predicting Drug Targets and Combinations

Abstract. Artificial intelligence-based approaches represent an opportunity for discovering new targets and drugs for human diseases. Here, DeepKinX, an integrated systems pharmacology and an interpretable neural networking framework, was developed and used this approach to identify novel protein kinase regulators, inhibitors, and synergistic drug combinations that decrease the viability of mesenchymal cancer cells. DeepKinX enables target deconvolution: the understanding of the molecular basis for the model's predictions.

Description. Advances in high-throughput drug profiling and large-scale molecular ‘omics’ data collection, coupled with exponentially improving computational power, have opened up an enticing avenue for applying artificial intelligence (AI)-driven methods to identify candidate ‘hit’ molecules¹. Physiology is regulated through complex, multifaceted signaling networks within and between cells. The complex and dynamic nature of biological data describing cellular responses underscores the need for computational Al models like Deep Neural Networks (DNNs)². Although such computational tools have been developed, a prominent obstacle in the field remains model interpretability³. As the model complexity increases, it is increasingly difficult to comprehend the underlying biological mechanisms through which neural networks (e.g., DNNs) reach their predictions, leading the models to be referred to as “black-box models”⁴. Consequently, simpler interpretable models are often more useful despite lower predictive accuracy over more complex models. To address this limitation, a neural network-based modeling framework, (FIG. 1A) was developed, that integrates and accurately model complex drug response data to predict the underlying molecular mechanisms behind the predictions. Knowledge of the molecular mechanisms is a pharmacologically imperative called ‘target deconvolution.’

The disclosed approach was applied to identify protein kinases essential for driving mesenchymal cancer cell state. As a cellular model system, the hepatocellular carcinoma (HCC) cell line, Huh7, was used, which has epithelial properties, and the Huh7 cells were engineered to overexpress FZD2 (Huh7-Fzd2), a receptor for Wnt5 that drives epithelial-mesenchymal transition (EMT)⁵⁻⁷. To generate the training set for model development, Huh7, and Huh7-Fzd2 cells were exposed to a panel of 44 computationally-chosen kinase inhibitors with known quantified effects against 298 human protein kinases⁸. Each inhibitor was examined at 8 concentrations, and the effect on cell viability was scored using CellTiter-Glo⁹. The quantitative inhibition profiles (Kinase profiling in FIG. 1A) were used and the cellular responses to each drug (Training set in FIG. 1A) to develop preliminary neural networks for both Huh7 (sometimes referred to herein as DeepKinX-Epi) and Huh7-Fzd2 (sometimes referred to herein as DeepKinX-Mes) were used. Using a multi-phase grid search, the hyperparameters for each preliminary neural network was optimized (Table 1, FIG. 2). Model performance was evaluated using leave one out cross validation (LOOCV) mean squared error (MSE) between predicted and observed drug response (See Equation 1 in Methods), as described³. LOOCV is one of the most widely used and effective methods in machine learning to evaluate overfitting. In LOOCV, each time 43 drugs' activity profiles are used to train the model to predict the remaining drug's effect on cell viability.

TABLE 1
Summary of Optimized Hyperparameters
for each of the DeepKinX models.

| Hyperparameter | DeepKinX-Epi | DeepKinX-Mes |
|---|---|---|
| Activation Function | ELU | ReLU |
| Batch Size | 2 | 44 |
| Dropout Rate | 0 | 0 |
| Epochs | 120 | 80 |
| Hidden Layers | 4 | 2 |
| Weight Initialization | Uniform | Truncated Normal |
| Nodes per Hidden Layer | 10 | 100 |
| Optimizer | Adamax | Adam |

To improve the models' predictive accuracies while simultaneously identifying which of the 298 kinases in the models were most important for establishing the epithelial or mesenchymal state, an iterative, model-agnostic method called recursive feature elimination was applied. Although permutation-based feature elimination has been applied to machine learning-based modeling¹⁰, its application to neural networks (e.g., DNNs) and biological data is relatively uncommon. In the modified permutation kinase importance (PKI) approach, first, an initial baseline error score (e_{baseline}) was assigned to each DeepKinX model based on the model's MSE of predicted and observed response on the original, unchanged 44-drug training matrix (see Equation 2 in Methods). Next, each feature, or kinase's activity, across all 44 drugs was shuffled 10,000 iterations while keeping the remaining matrix of features unchanged and was subsequently inputted into the DeepKinX model, tracking the MSE after each shuffle (eₚₑᵣₘᵤₜₐₜᵢₒₙ) (see Equation 3 in Methods). Each kinase was assigned relative kinase importance (RKI) score, which was calculated by subtracting the baseline MSE (e_{baseline}) from the MSE after permuting the feature (eₚₑᵣₘᵤₜₐₜᵢₒₙ), with higher RKI scores indicating greater reliance of the model on a specific kinase's activity. (see Equation 4 in Methods). By quantifying the models' error increases, it is possible to effectively compare the importance of various kinase's activity in contributing to the viability of epithelial or mesenchymal cancer cells. After ranking the kinases by RKI score, the bottom 25% of kinases were removed, and the top 75% of kinases were used to build a new DeepKinX model. The performance of the new model was tracked by LOOCV MSE (leaving out one inhibitor). The process was repeated of “recursive kinase elimination”—(1) ranking kinases, (2) removing the bottom 25% of kinases, and (3) assessing LOOCV MSE of the DeepKinX model built using the remaining 75% of kinases—until reaching an inflection point in the MSE (FIG. 1B). The LOOCV MSE DeepKinX-Epi was reduced from 176.7 to 30.1 after 9 iterations of recursive kinase elimination, and from 252.8 to 124.3 after 10 iterations for DeepKinX-Mes (FIG. 1B).

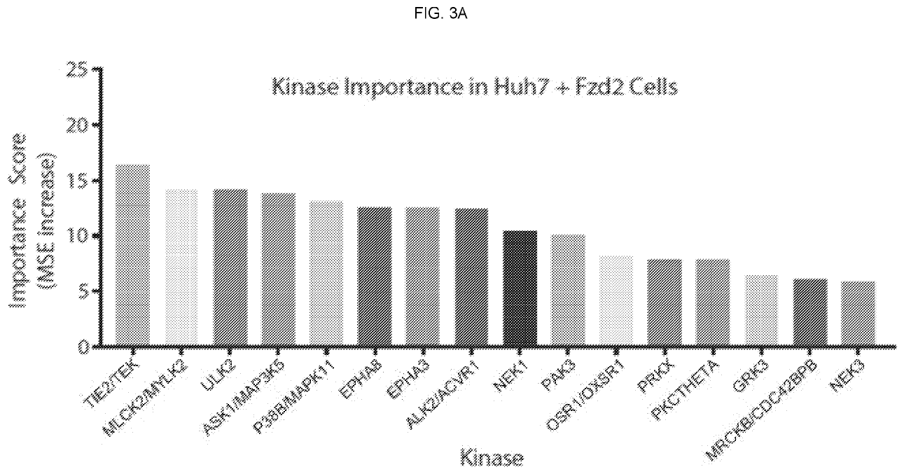

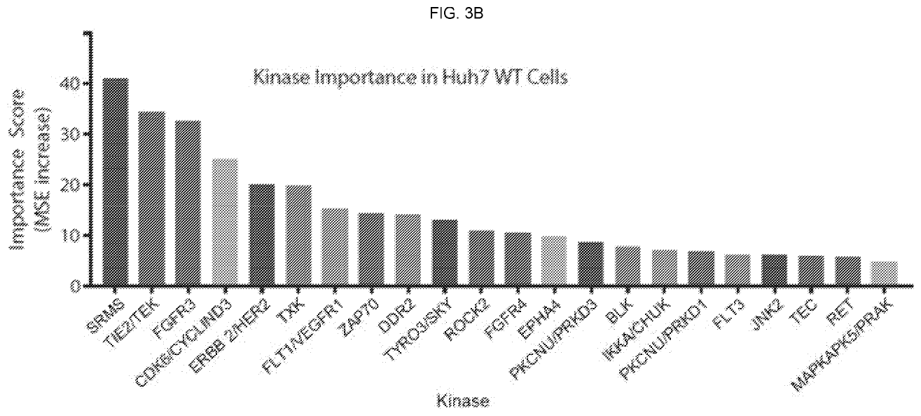

This process reduced the model's matrix of features from 298 kinases to 22 of the most important kinases in DeepKinX-Epi and 16 in DeepKinX-Mes (FIGS. 3A, 3B). These 22 and 16 kinases were used to build the final DeepKinX models, and their performance was assessed by LOOCV MSE. The strong correlation between observed and predicted drug response, 0.96 for DeepKinX-Epi and 0.87 for DeepKinX-Mes (FIG. 1C), indicated that recursive kinase elimination reduces the dimensionality of the input space through the identification of important kinases, while also improving the predictive performance of each model and minimizing overfitting (FIG. 1B).

To identify targets relevant for blocking the mesenchymal state, a selectivity score was determined for each of the 298 kinases by computing the difference in the rank-ordered lists of kinases based on the RKI scores both epithelial and mesenchymal models. The kinases were ranked in each model based on each kinase's relative ranking in each round of elimination until the inflection point in MSE (FIG. 1B). Using this strategy, 32 mesenchymal-selective kinases were identified, defined as having a selectivity score (epithelial RKI rank—mesenchymal RKI rank) greater than 150 (FIG. 1D). Overall, the disclosed framework enabled mechanistic insight into and target deconvolution of the neural networks.

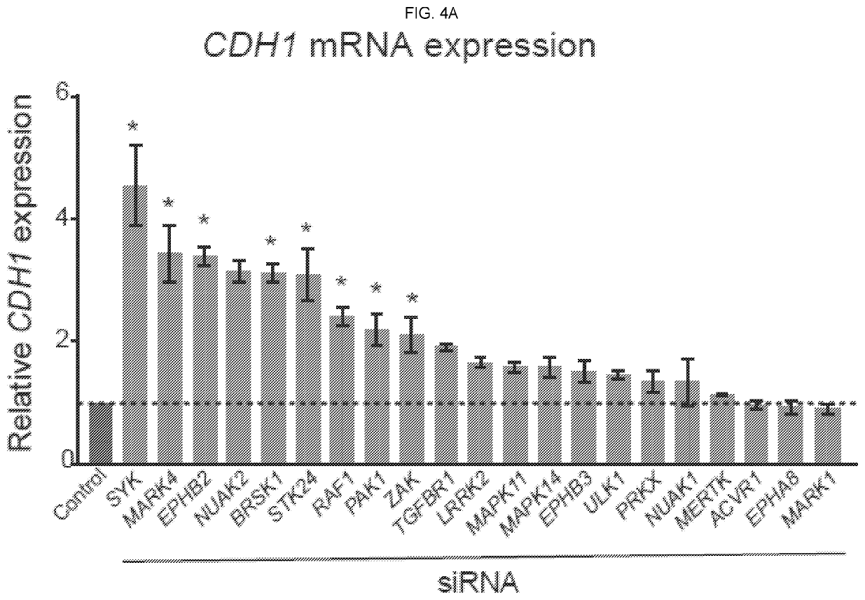

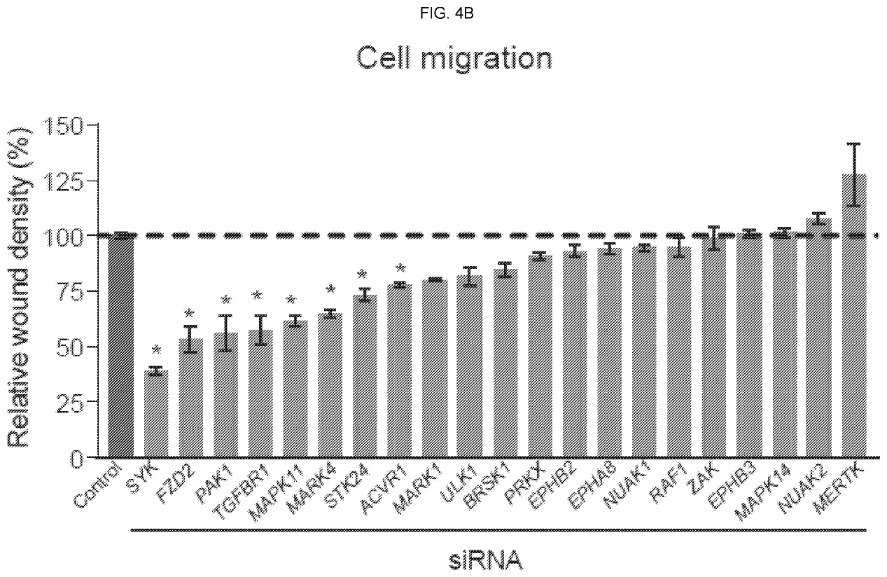

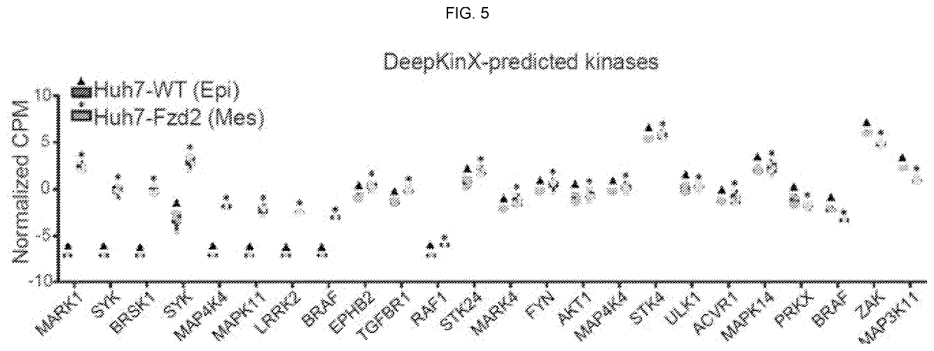

To validate the functional roles of the mesenchymal-selective kinases predicted by DeepKinX, 20 kinases were individually depleted in Huh7-Fzd2 cells by RNAi and assessed changes in the expression of CDH1 (encoding E-cadherin), a marker that is suppressed in mesenchymal cells, and in cell migration, properties of mesenchymal cells. Collectively, these results indicated that the 32 kinases included many that were important for these properties of mesenchymal cells (FIG. 4). Although the role of several of these kinases in EMT is well established, roles for BRSK1 and STK24 were not previously known. Consistently, many of the DeeepKinX-predicted mesenchymal-specific kinases were found, including BRSK1, are also overexpressed in mesenchymal Huh7-Fzd2 cells compared to parental epithelial-Huh7 cells (FIG. 5), further implicating the importance of these kinases in the mesenchymal cell state.

To determine the relevance of these findings for cancer, genes encoding the mesenchymal-selective kinases identified by DeepKinX were evaluated whether they are also expressed in mesenchymal-like tumors. The mRNA abundance of the 32 predicted kinases across 17 different cancer types in The Cancer Genome Atlas (TCGA) database were analyzed. Samples in TCGA were stratified into epithelial-like or mesenchymal-like states based on the expression of key marker genes7. The data showed that the expression of many of the genes for the top DeepKinX-predicted kinases, including BRSK1 and STK24, is significantly correlated with mesenchymal-type tumors across cancer types (FIG. 1E). These results indicated that the DeepKinX-predicted kinases mediate a mesenchymal state, both FZD2-driven cells and mesenchymal-like cancers.

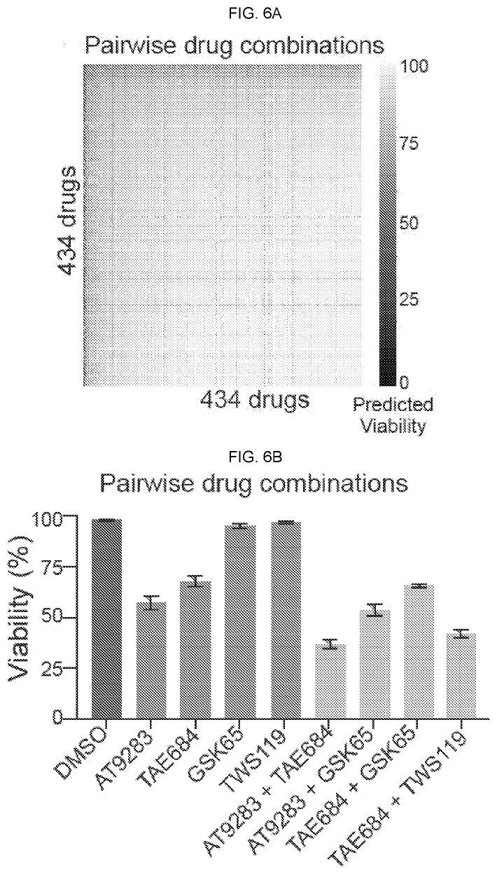

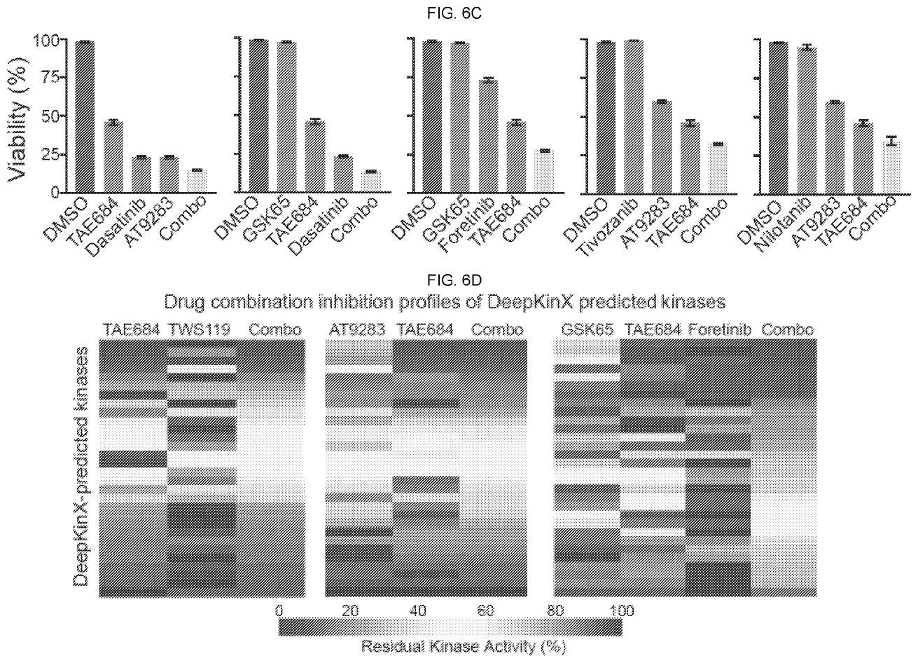

Like other approaches^{3, 11}, DeepKinX could be used to predict single-agent candidates; however, identifying combinations of inhibitors is likely more clinically useful¹². Therefore, the DeepKinX models were used to predict pairwise and three-drug combinations that reduce mesenchymal cancer cells' viability. A matrix of the predicted effect of 91,000 pairwise combinations of 427 single inhibitors (FIG. 6A) and of 13,000,000 three-drug combinations was generated. To limit experimental validation to combinations likely to exhibit synergistic effects, combinations were excluded containing the top 15 drugs predicted to be individually effective. Out of the remaining drug combinations, four pairwise combinations predicted to be effective and 5 three-drug combinations were experimentally evaluated. The viability of Huh7-Fzd2 cells exposed to each drug at 500 nM individually, at 250 nM in pairwise combinations, or at 165 nM in three-drug cocktails was assessed. All four pairwise combinations (FIG. 6B) and all five three-drug cocktails tested were equally or more effective than single-agent alone (FIG. 6C), indicating that the model predictions were accurate.

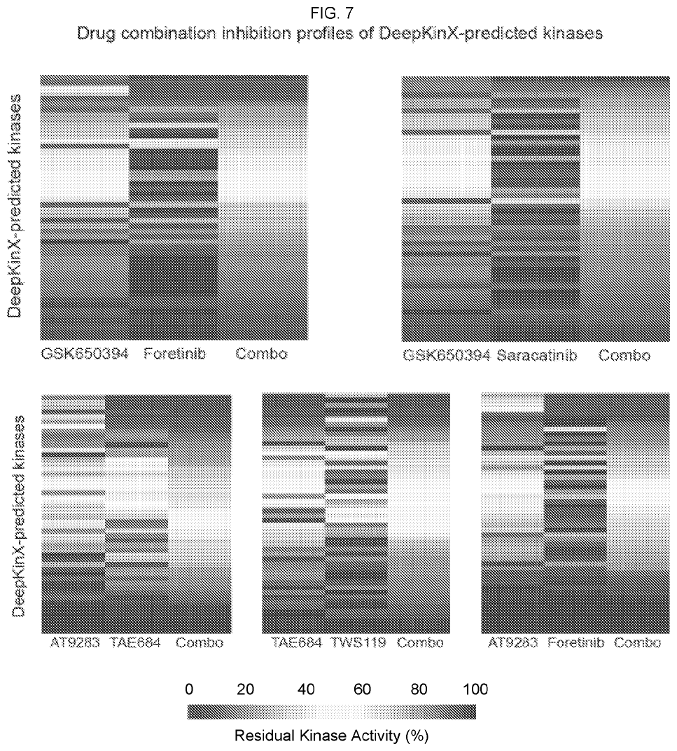

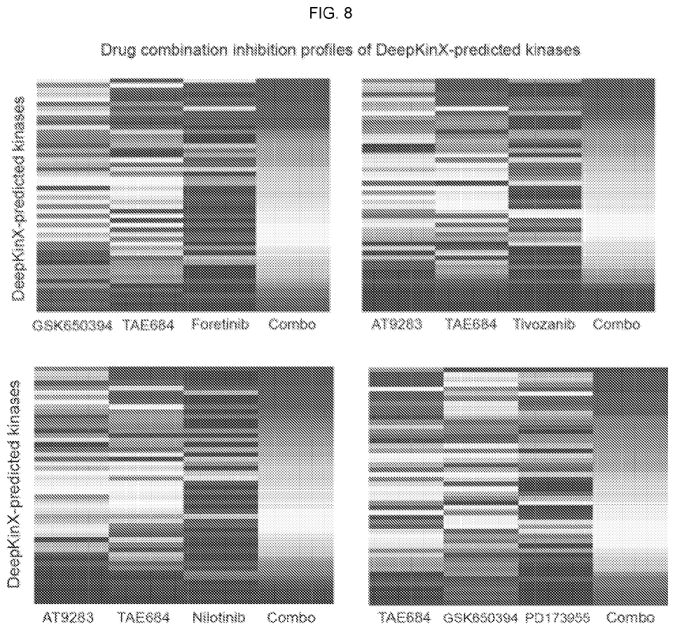

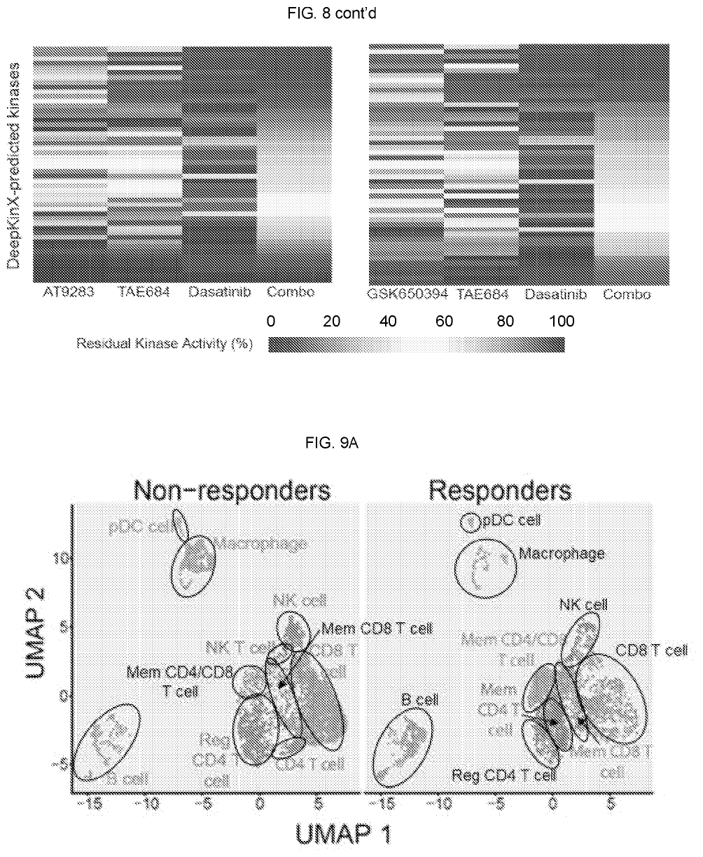

Some inhibitors predicted to exhibit synergy have overlapping target specificities, such as AT9283 and TAE684; others inhibit distinct targets, such as TAE684 and TWS119 (FIG. 6D). To understand the biological basis of the improved efficacy, the kinase inhibitor effects of selected combinations were compared with the predicted inhibitor effects of each drug individually for the top 30 (FIGS. 6D, 7, and 8) kinases ranked by RKI according to DeepKinX-Mes. In each case, a set of strongly inhibited kinases were identified in the combinations, providing leads to exploring biological mechanisms for the roles of these kinases in mesenchymal-like cell viability. Importantly, DeepKinX-identified effective drug combinations could be used to improve the computational design of molecular compounds and optimize in terms of mode of action and selectivity against specific kinases.

This framework can be applied to any neural network, not only kinases and their inhibitors and cell viability. The DeepKinX approach can be used to predict the effects from any dataset, such as drugs with known targets, protein knockdown by RNAi or targeted degradation, or gene knockout by CRISPR or other technologies, on molecular and phenotypic outcomes, using a training set of measurements. DeepKinX enables researchers to open the black box and reveal the underlying variables that are important for the predictions of the DNN.

Methods. Neural Network (e.g., DNN) Development. The development of the disclosed neural networks were achieved through the Keras and TensorFlow Deep Learning framework as described previously^{3, 13, 14}. Briefly, a multi-phase Grid Search method was used to optimize the DNN hyperparameters (epochs, batch size, optimizer, weight initializer, activation function, hidden layer quantity and nodes per hidden layer)¹⁵. Grid Search is a commonly employed method of hyperparameter optimization that evaluates combinations of numerous hyperparameter values to identify the model characteristics resulting in the lowest error between observed and predicted migration. The error function that was used to compare numerous models was LOOCV (Leave-One-Out-Cross-Validation) MSE¹⁶. In LOOCV, each time n−1 drugs' activity profiles are used to train the model to predict the remaining drug's effect on cell migration. The process is repeated n times, excluding and predicting each and every drug. Mean Squared Error (MSE) between predicted and observed migration is used to assign an error score to each model built with various combinations of hyperparameter values. In each phase of Grid Search, various combinations of hyperparameters are tested and the combination with the lowest LOOCV MSE is used in the subsequent phase of optimization until the final phase is reached. After optimization, the top performing hyperparameters are used to build the DeepKinX network.

Recursive Kinase Elimination (Mathematical Method). Assuming a trained neural network defined by f and a dataset defined by [y, X₁, X₂, X₃, . . . ], the baseline error (e_{baseline}) can be estimated, assuming a pre-defined cost function. For both DeepKinX-Fzd2 and DeepKinX-WT models, the cost function was defined as follows:

$$C ( M S E ) = \frac{1}{n} {\sum }_{i = 1}^{n} {( {y}_{i} - \hat{y} )}^{2} \qquad Equation 1$$

The baseline error was subsequently calculated as defined below:

$${e}_{baseline} = \frac{1}{n} {\sum }_{i = 1}^{n} C ( f ( {y}_{i} , {X}_{1 i} , {X}_{2 i} , {X}_{3 i} , … ) ) \qquad Equation 5$$

To calculate the post-permutation error (eₚₑᵣₘᵤₜₐₜᵢₒₙ), each feature may be shuffled one-by-one for a total of 10,000 random shuffles. In some examples, the shuffling may be determined systematically or, in another example, the shuffling may be random. The matrix of features with a single feature permuted once can defined by X^{permutation}. Accordingly, the post-permutation error for an individual feature is computed as follows:

$${e}_{permutation} = \frac{1}{10 , 000} {\sum }_{j = 1}^{10 , 000} \frac{1}{n} {\sum }_{i = 1}^{n} C ( f ( y , {X}_{i}^{permutation} ) ) \qquad Equation 6$$

Lastly, the relative kinase importance (RKI) or error difference for an individual feature is computed:

$$RKI = {e}_{permutation} - {e}_{baseline} \qquad Equation 7$$

Each kinase is then assigned an RKI score and ranked based from highest to lowest. Subsequently, the bottom 25% of kinases are removed in future iterations of recursive kinase elimination. Using just the top 75% of kinases, a new DeepKinX model is built and LOOCV MSE is used to track the model's overall relative performance across several rounds. This three-step process—(1) ranking kinases by importance score, (2) removing the bottom 25% of kinases, and (3) assessing LOOCV MSE of the DeepKinX model built using only the remaining kinases—is repeated until the LOOCV MSE of the model reaches an inflection point and starts to increase as the number of inputs decrease.

Selectivity score. To calculate kinases that were important in the mesenchymal cell model (DeepKinX-Mes) but not in the epithelial cell model (DeepKinX-Epi), a selectivity score was computed by subtracting the epithelial RKI rank from the mesenchymal RKI rank for all 298 kinases.

Prediction of naïve drugs and drug combinations. To predict the effect of all 427 drugs of the original matrix, the final DeepKinX-Huh7 model was trained on the 44-drug training matrix. Keeping the weights and biases of the DeepKinX-Huh7 network constant, new kinase activity data for naïve drugs were inputted into the model for prediction. Further, the effect of combinations of drugs were also predicted by creating pseudo-activity matrices. Assuming two activity matrices for two different drugs defined by [J₁, J₂, J₃, . . . Jₙ] and [K₁, K₂, K₃, . . . Kₙ], where n corresponds to a specific kinase's inhibition, a linear combination of the two activities corresponding to each drug for each kinase was applied to create a pseudo-activity matrix (P) combining both drug's effect on a kinase:

$${P}_{n} = \frac{{J}_{n} {K}_{n}}{{J}_{n} + {K}_{n} - {J}_{n} {K}_{n}} \qquad Equation 8$$

Subsequently, the pseudo-matrix for all 428 by 428 (including control) combinations of drugs was computed and inputted into DeepKinX for prediction. Because combinations of drugs that are effective in combination but not as effective individually are of particular interest, the top 15 drugs predicted individually are removed from the rank-ordered list of predicted viability of all drug combinations. The process of pseudo-matrix creation and successive prediction was similarly extended to 3 drug combos, in which a linear combination of all 3 residual kinase activities for each of 3 drugs was used.

Cell lines and reagents. Hepatocellular Huh7 cells were obtained from American Type Culture Collection. Stable Huh7 cell line expressing Fzd2 has been described previously⁶. Both cell lines were grown at 37° C. under 5% CO2, 95% ambient atmosphere and maintained in Dulbecco's minimum essential medium supplemented with 10% FBS (Sigma) and 1% Penn Strep.

Kinase inhibitor screening. Kinase inhibitor screening was performed as described previously⁶. Briefly, 42 kinase inhibitors were tested for the effect on cell growth and viability at 6-8 different concentrations in Huh7 parental and Huh7 cells expressing Fzd2 using real-time microscopy using Incucyte imaging system (Sartorius). The percentage viability at 500 nM calculated using the full-dose response curves for each of the inhibitors was used as a response variable for DeepKinX modeling.

Cell viability. The effects of inhibitors as single agent and in combinations on viability of Huh7-Fzd2 cells was measured using both live cell imaging (Incucyte Zoom, Ann Arbor, MI) and CellTiter-Glo assay (Promega, WI, USA) as described previously^{11, 17}. Briefly, cells (5×10³ in 100 ul culture medium) were seeded on a 96-well plate (Corning, NY, USA). Next day, cells were treated with various inhibitors at 500 nM as single agent, or 250 nM in two-drug combination or at 166 nM in three drug combination and placed in IncuCyte for imaging every 2 hours. After 4 days, cells were incubated with CTG2.0 reagent for 5 min and total viability was measured by obtaining luminescent signal intensity. The quantified data was normalized to untreated control and plotted in Prism (Graphpad software, San Diego, CA, USA).

Small interfering RNA transfection. All small interfering RNA (siRNA) were obtained from Dharmacon (Thermo). siRNA's transfections in both 12-well plate for expression profiling and in 96-well plates for cell migration were carried out using Lipofectamine RNAiMax (Invitrogen) according to manufacturer instructions.

RNA extraction and quantitative PCR. Total cellular RNA was isolated using an RNeasy Mini Kit (QIAGEN). mRNA expression changes in CDH1 (E-cadherin) was determined using quantitative real-time PCR (qPCR). Briefly, 1 μg of total RNA was reverse transcribed into first-strand cDNA using an RT2 First Strand Kit (QIAGEN). The resultant cDNA was subjected to qPCR using human CDH1-specific primer (Realtimeprimers.com) and GAPDH (housekeeping control). The qPCR reaction was performed with an initial denaturation step of 10 min at 95° C., followed by 15 s at 95° C. and 60 s at 58° C. for 40 cycles using Biorad CFX384 thermocycler (Biorad). The mRNA levels of CDH1 were normalized relative to the mean levels of the housekeeping gene and compared using the 2-ΔΔCt method as described previously⁶.

Cell migration assay. To study the role of DeepKinX-predicted kinases in cell migration, a wound-healing assay was employed as described previously⁶. Briefly, siRNAs targeting various proteins and scrambled control were transfected in Huh7-Fzd2 cells using Lipofectamine RNAiMax (Invitrogen) according to manufacturer instructions. Cells were plated on 96-well plates (Sartorius) and 48 hours post transfections, a wound was scratched with wound scratcher (Sartorius). Wound confluence was monitored with Incucyte Live-Cell Imaging System. Wound closure was observed every 2 hours for 24-72 hours by comparing the mean relative wound density of at least three biological replicates.

TCGA data analysis. Patient data and clinical manifests were downloaded from selected TCGA (The Cancer Genome Atlas) projects using the GenomicDataCommons Bioconductor package in R. Seventeen TCGA patient cohorts, containing 7881 patients in total, were selected, representing both high incidence and highly aggressive cancer subtypes. Data was processed as described previously⁷. To assess co-expression between DeepKinX predicted mesenchymal-cell specific kinases, previously reported epithelial and mesenchymal marker genes were used to rank patients in each TCGA cohort by calculating the mean-rank of their epithelial marker expression and mesenchymal marker expression, giving an E-score and M-score respectively. Patients with above median E-score and below median M-score were labelled as the epithelial-like group, while patients with above median M-score and below median E-score were labelled as mesenchymal-like. LogCPM of the high-value signature was calculated by taking the average of the log (CPM+1) values for all available genes within each sample. P-values were calculated using a Wilcoxon rank-sum test. The comprehensive list of cancer types analyzed is as follows: breast invasive carcinoma, cervical squamous cell carcinoma and endocervical adenocarcinoma, colon adenocarcinoma, glioblastoma multiforme, head and neck squamous cell carcinoma, kidney renal clear cell carcinoma, liver hepatocellular carcinoma, lung adenocarcinoma, lung squamous cell carcinoma, mesothelioma, ovarian serous cystadenocarcinoma, pancreatic adenocarcinoma, prostate adenocarcinoma, rectum adenocarcinoma, sarcoma, skin cutaneous melanoma, and stomach adenocarcinoma.

Data availability. The primary matrix used to train the DeepKinX model is available at https://github.com/sidvijay10/DeepKinX EMT. The Huh7-Fzd2 mass spectrometry dataset generated in this study are available online in the MassIVE repository (https://massive.ucsd.edu/) under the dataset ID MSV000086446. Patient data and clinical manifests were downloaded from selected TCGA (The Cancer Genome Atlas) projects through the National Cancer Institute's Genomic Data Commons data portal (https://portal gdc.cancer.gov/) using the GenomicDataCommons Bioconductor package in R.

Code availability. The custom python scripts that implements DeepKinX framework are available on GitHub: https://github.com/sidvijay10/DeepKinX EMT. This repository also includes all associated files needed to execute the script and produce a sample model using the training dataset.

### REFERENCES FOR EXPERIMENTAL EXAMPLE 1 & ASSOCIATED FIGURE DESCRIPTIONS

1. Ward, R.M., Schmieder, R., Highnam, G. & Mittelman, D. Big data challenges and opportunities in high-throughput sequencing. Systems Biomedicine 1, 29-34 (2013).
2. Angermueller, C., Pärnamaa, T., Parts, L. & Stegle, O. Deep learning for computational biology. Mol Syst Biol 12, 878 (2016).
3. Vijay, S. & Gujral, T.S. Non-linear Deep Neural Network for Rapid and Accurate Prediction of Phenotypic Responses to Kinase Inhibitors. Iscience 23 (2020).
4. Vellido, A. The importance of interpretability and visualization in machine learning for applications in medicine and health care. Neural Computing and Applications (2019).
5. Golkowski, M. et al. Pharmacoproteomics Identifies Kinase Pathways that Drive the Epithelial-Mesenchymal Transition and Drug Resistance in Hepatocellular Carcinoma. Cell Syst 11, 196-207 e197 (2020).
6. Gujral, T.S. et al. A noncanonical Frizzled2 pathway regulates epithelial-mesenchymal transition and metastasis. Cell 159, 844-856 (2014).
7. Xue, A.G., Chan, M. & Gujral, T.S. Pan-cancer analysis of the developmental pathways reveals non-canonical wnt signaling as a driver of mesenchymal-type tumors. Transl Res 224, 1-15 (2020).
8. Rata, S. et al. An optimal set of inhibitors for Reverse Engineering via Kinase Regularization. bioRxiv (2020).
9. Gujral, T.S. & Kirschner, M.W. Hippo pathway mediates resistance to cytotoxic drugs. Proc Natl Acad Sci U S A 114, E3729-E3738 (2017).
10. Putin, E. et al. Deep biomarkers of human aging: application of deep neural networks to biomarker development. Aging (Albany NY) 8, 1021 (2016).
11. Gujral, T.S., Peshkin, L. & Kirschner, M.W. Exploiting polypharmacology for drug target deconvolution. Proc Natl Acad Sci U S A 111, 5048-5053 (2014).
12. Al-Lazikani, B., Banerji, U. & Workman, P. Combinatorial drug therapy for cancer in the post-genomic era. Nature Biotechnology 30, 679-692 (2012).
13. Chollet, F. (2015).
14. Abadi, M. et al. Tensorflow: Large-scale machine learning on heterogeneous distributed systems. arXiv preprint arXiv: 1603.04467 (2016).
15. Bergstra, J.S., Bardenet, R., Bengio, Y. & Kégl, B. in Advances in neural information processing systems 2546-2554 (2011).
16. Zhang, P. Model selection via multifold cross validation. The annals of statistics, 299-313 (1993).
17. Vijay, S. & Gujral, T.S. Non-linear Deep Neural Network for Rapid and Accurate Prediction of Phenotypic Responses to Kinase Inhibitors. iScience 23, 101129 (2020).

### Example 2. Deep Neural networking Identifies Biomarkers of Response to Immune-checkpoint Therapy

Abstract. Immunotherapy has shown significant promise as a treatment for cancer, such as lung cancer and melanoma. However, only 10-30% of the patients respond to treatment with immune checkpoint blockers (ICBs), underscoring the need for biomarkers to predict response to immunotherapy. Here, DeepGeneX was developed, a computational framework that uses advanced deep neural networking and feature elimination to reduce single-cell RNA-seq data on 26,000 genes to six of the most important genes (CCR7, SELL, GZMB, WARS, GZMH, and LGALS1) that accurately predict response to immunotherapy. It was also discovered that the high LGALS1 and WARS-expressing macrophage population represent a biomarker for ICB therapy non-responders, suggesting that these macrophages may be a target for improving ICB response. Taken together, DeepGeneX enables biomarker discovery and provides an understanding of the molecular basis for the model's predictions.

Introduction. In order to prevent the immune system from destroying cells indiscriminately, T cells use immune checkpoints mediated by immune checkpoint molecules. These molecules are membrane receptors classified as either stimulatory or inhibitory, depending on whether they activate or inhibit T cell response. Many tumors have strategies to evade immunosurveillance by expressing ligands for inhibitory immune checkpoint molecules, such as programmed cell death 1 (PD1) and cytotoxic T lymphocyte-associated protein 4 (CTLA4), which prevents T cells from recognizing and destroying tumor cells. Recently, drugs that block these interactions, called immune checkpoint blockers (ICBs), have emerged as effective therapies for cancer, especially melanoma and lung cancer (Waldman et al., 2020). In comparison to conventional cancer treatments, such as chemotherapy and radiotherapy, which harm the immune system due to their untargeted (systemic) effects, ICBs was shown to be more specific and restrained, with a significant enhancement in the patients' survival (Esfahani et al., 2020); (Dwary et al., 2017; Vera Aguilera et al., 2020). However, despite these benefits, clinical data highlight that ICBs are not universally effective, as only 10-30% of patients that receive ICBs respond to treatment (Ventola, 2017). Furthermore, as these agents activate the immune response, they pose a risk for triggering a severe auto-immune response (Staff, 2019). These deficiencies highlight the need for strategies to identify patients who will respond to the ICB therapy and unravel physiological and mechanistic differences between the responders and non-responders.

To address these challenges, numerous research efforts have been directed towards the discovery of predictive biomarkers of the positive therapeutic effect (Bai et al., 2020). Currently, parameters such as tumor mutational burden, the status of DNA damage response pathways, and tumor immune microenvironment, as well as liquid biopsy biomarkers, including circulating tumor DNA, have been investigated and/or adopted as a part of clinical practice (Bai et al., 2020). An emerging strategy for predicting ICB response and investigating the molecular basis for clinically observed differences between responders and non-responders is single-cell RNA sequencing (scRNA-seq), i.e. single-cell transcriptomics. Previously, scRNA-seq analysis of CD8 T cell population from a large cohort of melanoma patients treated with ICBs led to the identification of TCF7, a gene coding for a transcription factor involved in T cell differentiation, as a biomarker that correlates with ICB therapy (Sade-Feldman et al., 2018). Although this study presented promising findings, they relied on simplistic linear correlational models. Furthermore, they focused on CD8 T cells, thus disregarding the impact of the tumor microenvironment (TME), which drives multiple aspects of tumorigenesis, including response to therapy (Binnewies et al., 2018). More recently, bulk RNA-seq data across 18 solid cancers from more than 7,500 patients, combined with a range of prior biological knowledge, was used to develop a machine learning model to construct systems-level signatures predictive of ICB response (Lapuente-Santana et al., 2021). However, given their complexity, systems biomarkers may be challenging to interpret and act upon in routine clinical practice.

To address the need for easy-to-use, clinically actionable biomarkers of ICB response that take into account features of the TME, a new analytical framework was developed, DeepGeneX. DeepGeneX uses sc-RNA-seq data, advanced deep neural networking, and feature elimination steps to identify a smaller set of genes that could predict a patient's immune response to ICB therapy. DeepGeneX models outperformed linear models and identified a set of six genes that could predict the response to ICB in melanoma with 100% accuracy. The expression of these marker genes was further examined in different types of immune cells in the TME and identified two genes, LGALS1 and WARS, that expressed significantly higher in macrophages of non-responders compared to those of responders. Gene set enrichment and cell-cell interaction analysis suggest the macrophages with high expression of LGALS1 and WARS (MΦ^{LW}-high) are immunosuppressive and could directly promote T cell exhaustion and suppress T cell function. Thus, the MΦ^{LW}-high macrophage population not only represents a biomarker for ICB therapy but targeting this population could enhance ICB therapy in non-responders.

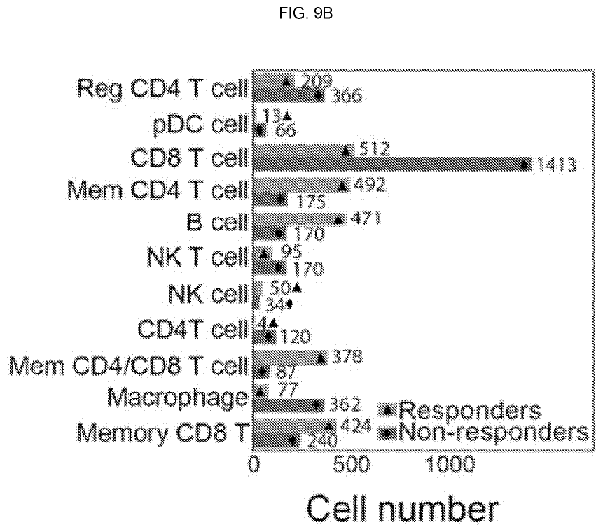

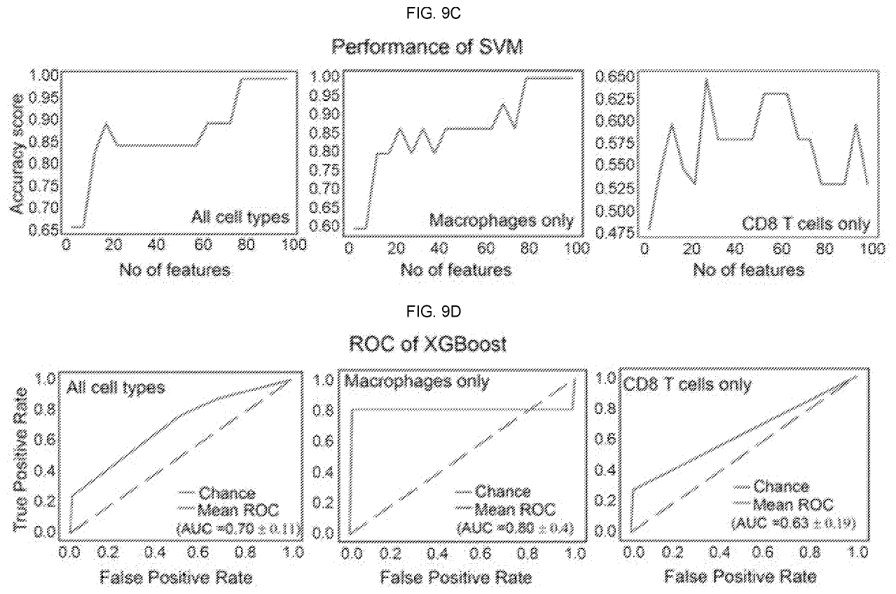

Results. Modeling sc-RNA-seq data to identify molecular drivers of response to immunotherapy. It was reasoned that the interactions between T cells and other cells in the TME could affect immune checkpoint therapy response. To model and identify molecular signatures from the broader tumor immune microenvironment (TIME) that could predict responses to immunotherapy, a recently published sc-RNA-seq dataset was used from melanoma patients treated with various immune checkpoint therapy (Sade-Feldman et al., 2018). First, the distribution of different immune cells was analyzed in the stroma from responders and non-responders and found a two-fold higher number of CD8 T cells and a four-fold higher number of macrophages in non-responders than the responders (FIGS. 9A, 9B). In addition, most CD4 T cells, which are known to correlate with poor clinical outcomes, were also observed in higher frequency in non-responders (Pan et al., 2020) (FIGS. 9A, 9B). These observations are consistent with the previous study (Sade-Feldman et al., 2018) and suggest that increase in the myeloid/macrophage population may suppress or cause exhaustion of CD8 T cells in non-responders.

To identify molecular markers of immune checkpoint therapy response, naïve predictive modeling was applied to the data from all cells in the tumor or macrophages or CDT cells. Specifically, the support vector machine (SVM) and XGBoost were applied, to distinguish the responder and non-responder population using the immune cell gene expression data. The SVM classifies patients as responders or non-responders based on drawing a plane to separate patients into two classes, while XGBoost adapts a decision-tree algorithm that separates patients with each branching and assigns a label (response or not) at the final leaf node. The data show that SVM required the expression data from over 80 genes to accurately predict the outcome from all immune cell populations and macrophages (FIG. 9C). In addition, the SVM failed to perform better than a random guess when CD8 T cell gene expression data (FIG. 9C). Similarly, the ROC curves from the XGBoost modeling indicated either an unreliable prediction (low average AUC value) or an unstable one (sharp change in ROC curve) (FIG. 9D), suggesting a more complex and non-linear modeling approach is warranted to predict patient outcome accurately.

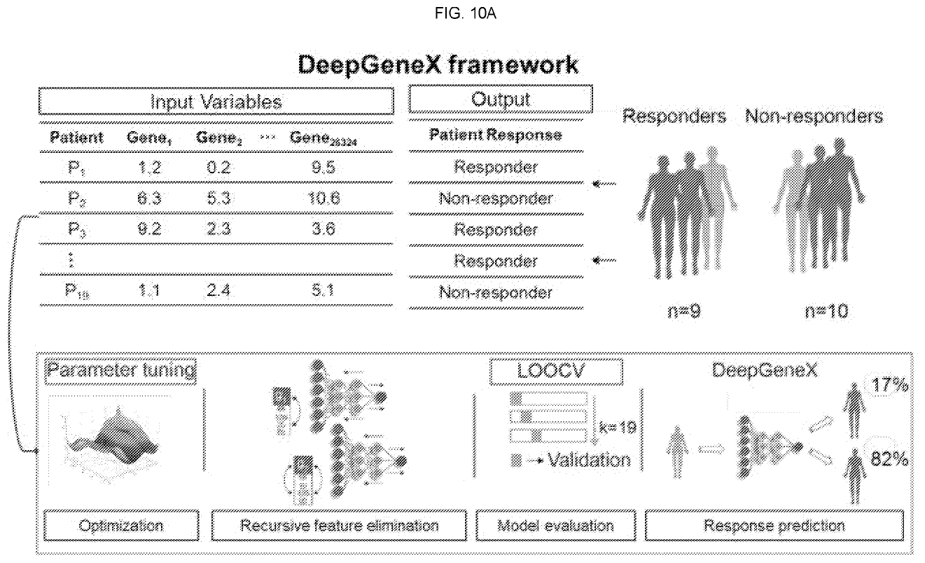

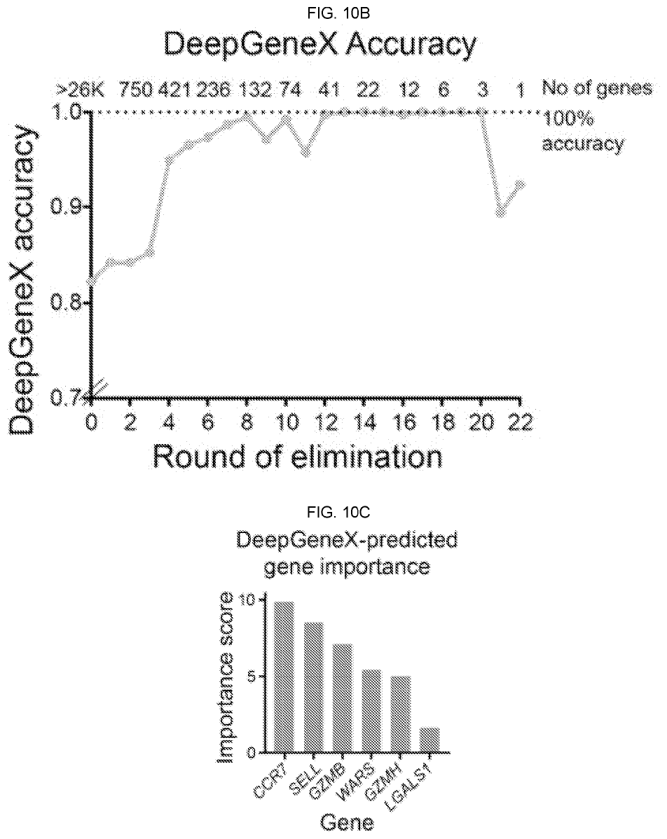

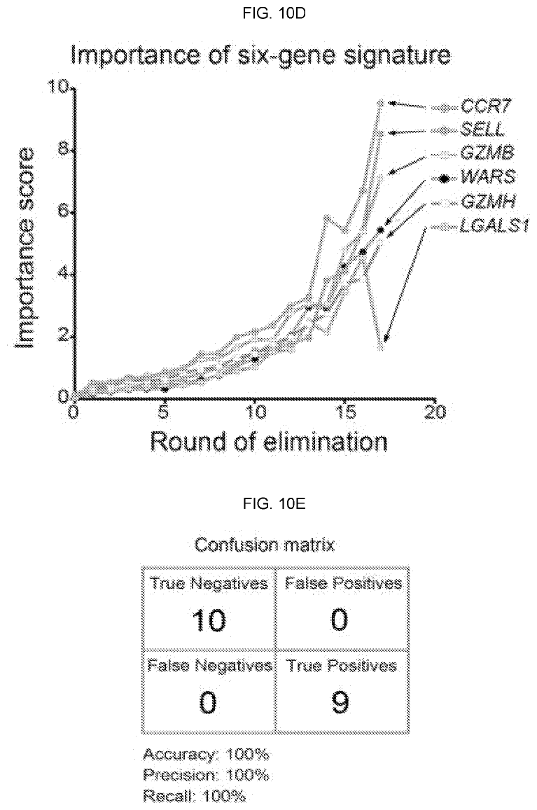

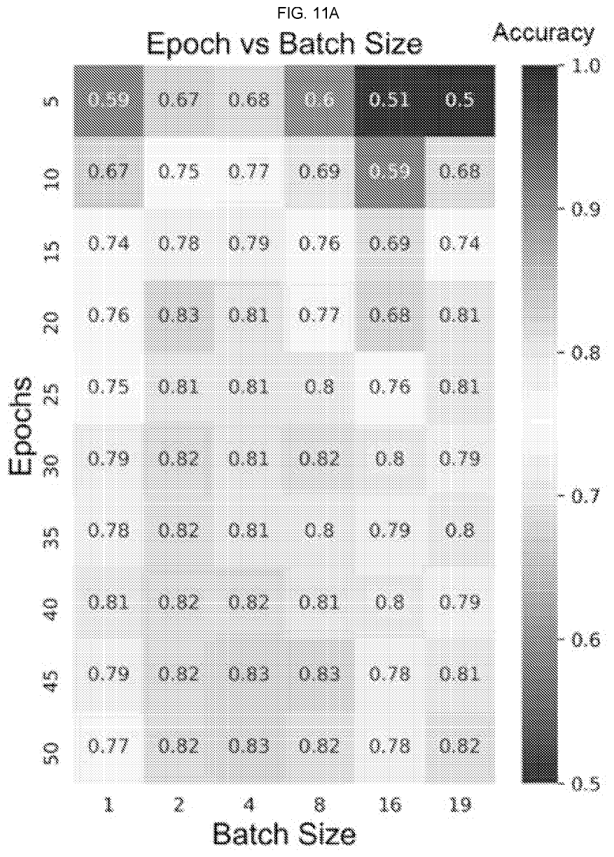

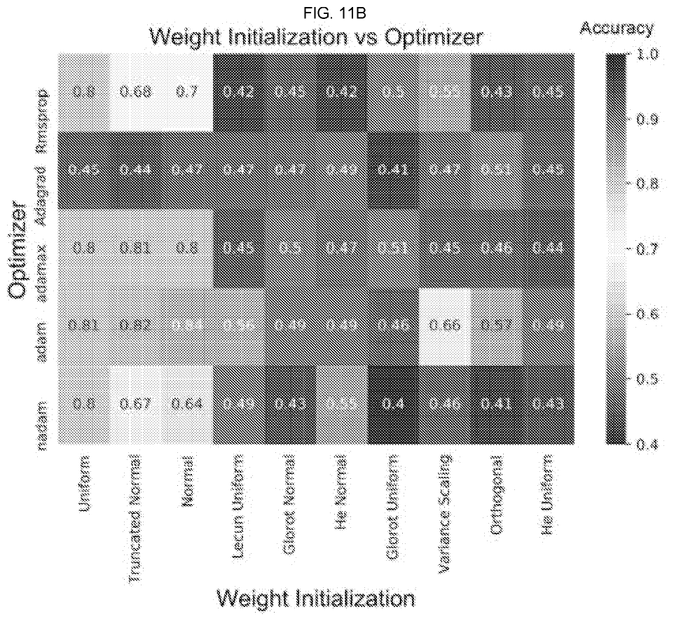

Deep Neural Networks identifies genesets that can predict patient response. Another shortcoming of XGBoost models is that they may not perform well on large datasets. Given that the data measures the activity of more than 26,000 genes, it was hypothesized that a deep neural network architecture might model the large dataset better. Deep neural network (DNN) modeling was explored to identify biomarkers of immune checkpoint therapy response using data from all immune cells. Neural networks (e.g., DNNs) are non-linear models that are analogous to neurons in the human brain (Zupan, 1994). Neural networks have an input layer, output layer, and hidden layers in between connected by weighted links that capture complex relations in data. Neural networks have previously been applied to biological modeling, including proteomic, genomic, and other high-throughput data (Grapov et. al, 2018). The neural network was built through several stages, as conceptualized in FIG. 10A.

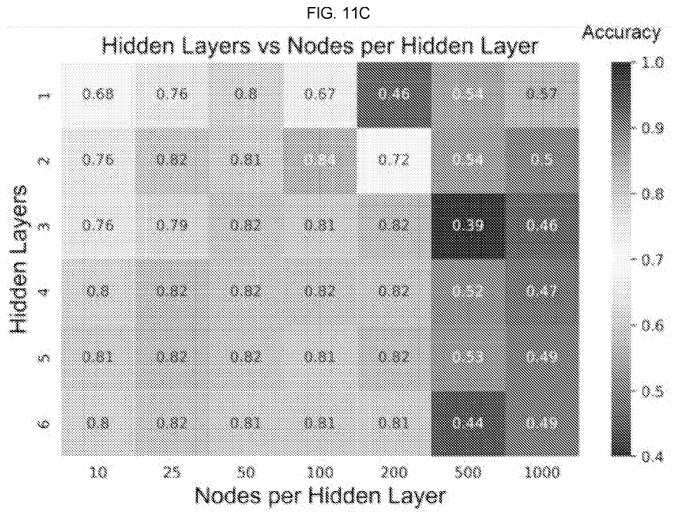

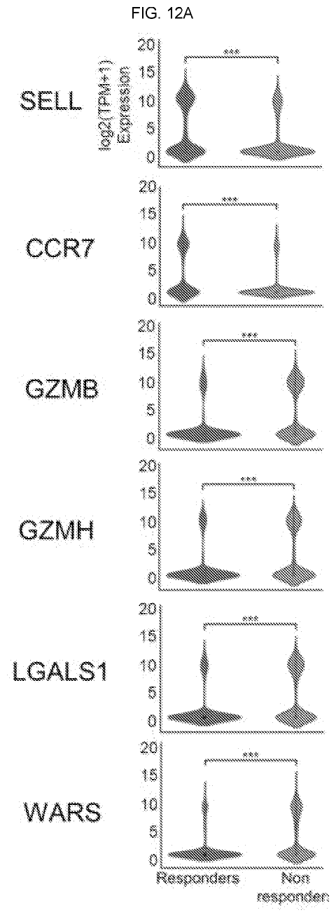

To build neural networking of the sc-RNA-seq data, a multi-stage Grid Search method was first used to optimize the model hyperparameters. Next, model performance was evaluated using leave-one-out cross-validation (LOOCV) binary cross-entropy between predicted and observed responses to the immune checkpoint inhibitor. In LOOCV, a dataset was used that contains information on 19 patients in total, and at each step, data from 18 patients were used to train the model to predict the remaining patient's responses. The Grid Search method was used to search through hundreds of combinations of hyperparameters to identify a single combination with the lowest LOOCV binary cross-entropy cost. In each round of optimization, two hyperparameters were tuned: epoch and batch size, weight initializer and optimizer, and hidden layers and nodes per hidden layer in rounds 1, 2, and 3, respectively (FIG. 11). The resulting optimized network involved 2 hidden layers with 100 nodes per layer, the normal weight initialization, exponential linear unit (elu) activation function and the Adam optimizer. The model was trained for 45 epochs with a batch size of 4. The average accuracy of the model was 0.82 in LOOCV.

Next, the aim was to improve the model's predictive accuracy while also identifying which of the 26,000 genes in the model were indicative of ICB response. A method called “permutation gene importance” (PGI) was employed. First, an initial baseline error score was assigned to the neural network based on the model's binary cross-entropy error of predicted response on the original, unchanged 19 patient training matrix. Subsequently, each gene's activity was shuffled across all 19 patients while keeping the remaining matrix of features unchanged and inputted the data into the neural network, tracking the binary cross-entropy error after each shuffle. Each gene was assigned a “gene importance” score which was calculated by subtracting the baseline binary cross-entropy error from the error after permuting the feature. By measuring the model's error changes, the importance of different gene's activity in contributing to a positive or negative response of the patient was estimated, with higher error changes (i.e. gene importance scores) indicating greater reliance of the model on that specific gene's activity. From this, a ranked list of the most important genes was obtained. After ranking the genes by importance score, the top 1000 genes was used to build a new model. Having filtered the genes with only the top 1000 genes, the process of recursive gene elimination was iteratively used—(1) ranking genes by importance score, (2) removing the bottom 25% of genes, and (3) assessing LOOCV binary cross-entropy of the neural network built using the remaining 75% of genes (FIG. 10B).

Applying the above strategy led to significant improvement in the model's accuracy from 0.82 to 1.0 over several rounds of gene elimination (FIG. 10B). Seven gene sets (all<10 genes) were identified that resulted in a 100% LOOCV accuracy. Of these, a six gene set signature was chosen for further exploration. This set includes CCR7, SELL, GZMB, WARS, GZMH, and LGALS1, in order of predicted importance (FIG. 10C). Overall, the process of permutation gene importance reduced the model's matrix of features from 26,000 genes to 6 of the most important genes. The importance scores of these six genes in each round of elimination are shown in FIG. 10D. These six genes were used to build the final neural network (sometimes referred to herein as DeepGeneX), and its performance was assessed by a confusion matrix and LOOCV accuracy, precision, and recall—all of which were 100% (FIG. 10E). These model performance indicators suggested that the disclosed model effectively predicted whether a patient is a responder or non-responder and that recursive gene elimination could reduce the dimensionality of the input space to identify gene signatures that were indicative of immune checkpoint response.

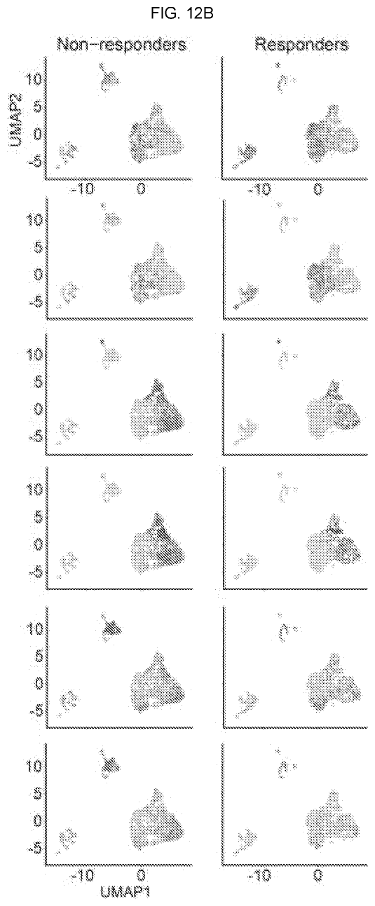

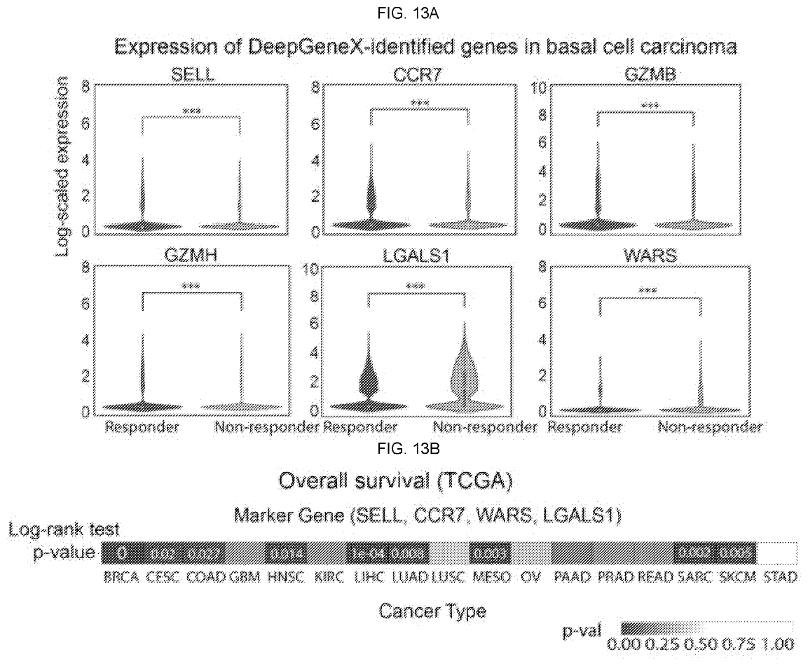

Identified marker genes are differentially expressed in responders and non-responders. To gain biological insights into the marker genes identified by disclosed neural networks, the expression pattern of six marker genes was next analyzed in the sc-RNAseq data from responders and non-responders. The data show that all six genes were differentially expressed between responders and non-responders (FIG. 12A). SELL and CCR7 were expressed at significantly higher levels in responders, while GZMB, GZMH, LGALS1, and WARS expression in responders was significantly lower (FIG. 12A). Further, differential expression of these marker genes was also observed in specific immune cell types. Consistent with previous studies (Martin and Badovinac, 2018; Sade-Feldman et al., 2018), the predominant expression of SELL and CCR7 was observed in memory T cells. These genes were also expressed in a more significant proportion of memory T cells in responders compared with non-responders. In contrast, GZMB, and GZMH, known to be expressed in cytotoxic cells (Hashimoto et al., 2019), were mainly expressed in the NK cells and CD8 T cells iof non-responders (FIG. 12A). Lastly, LGALS1 and WARS were expressed primarily in the macrophages of non-responders, showed a significant correlation (co-efficient=2.75, p-value=0.00014). Previous studies have shown that LGALS1 plays an essential role in promoting the differentiation of M2-like macrophage and therefore driving an immunosuppressive TME (Abebayehu et al., 2017; Chen et al., 2019). Similarly, WARS was shown to induce the secretion of a panel of Interferons (IFN) and cytokines, which contributed to the activation of tumor-associated macrophages (TAMs) (Brown et al., 2010; Nie et al., 2019). Thus, based on the higher expression of LGALS1 and WARS in the macrophages of the non-responder population and the previously documented role of these genes, it is posited that high LGALS1 and WARS expressing TAM population may negatively impact response to immune checkpoint therapy.

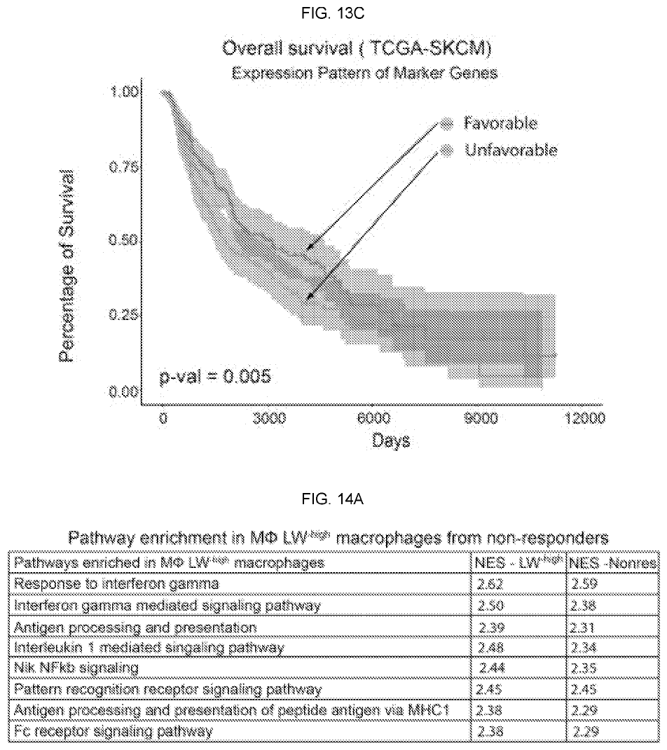

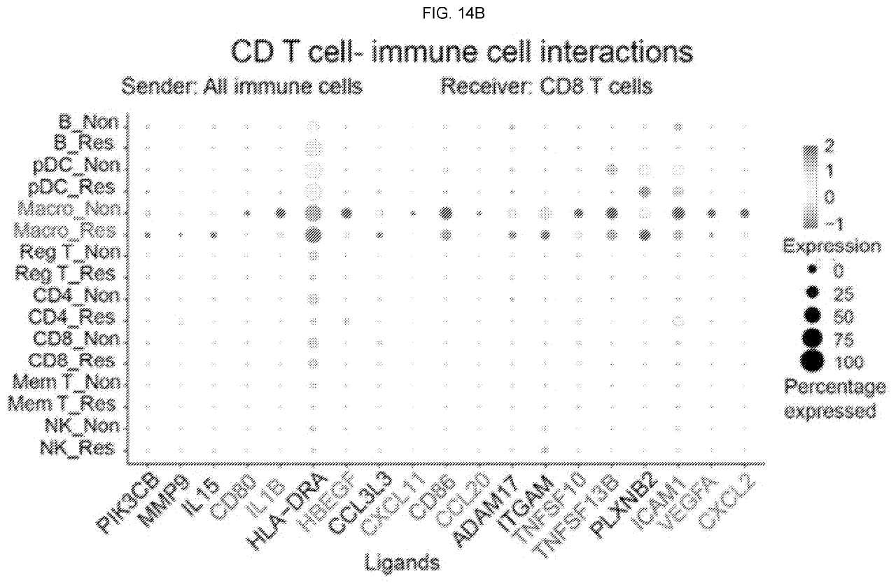

Validating predicted marker genes across cancer types. To determine whether identified marker genes could predict response to immunotherapy in other cancers, the differential expression was compared between responders and non-responders using a scRNA-seq dataset from basal cell carcinoma patients (Yost et al., 2019). Four out of six genes (SELL, CCR7, LGALS1, and WARS) showed a consistent trend with immunotherapy response. As predicted, significantly higher expression of SELL and CCR7 were found in responders and substantially higher expression of LGALS1 and WARS in non-responder in basal cell carcinoma patients (FIG. 13A). To further extend the validity and generality of defined marker genes, the expression pattern of four genes and patients' overall survival across seventeen cancer types using the bulk RNA-seq data and clinical data from The Cancer Genome Atlas (TCGA) was then assessed (FIG. 13B). The data show that patients with the favorable expression pattern of marker genes (high expression in SELL/CCR7 and low expression in LGALS1/WARS) showed significantly better survival rates compared to those with unfavorable gene expression patterns (Log-rank test, p-value <0.05) in nine out of seventeen cancer types, including SKCM (Skin Cutaneous Melanoma) (FIGS. 13B, 13C). Together, these orthogonal analyses suggest that the applicability of the identified marker gene set is not limited to skin melanoma cancer type or sc-RNA-seq data.

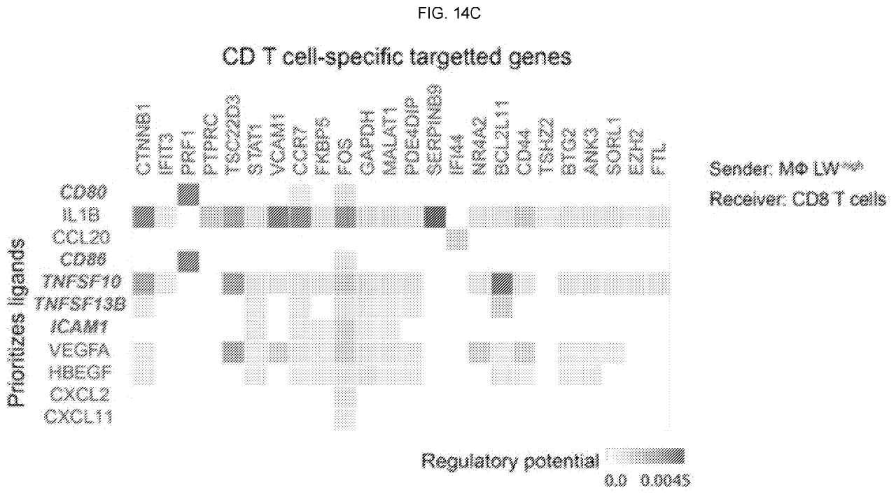

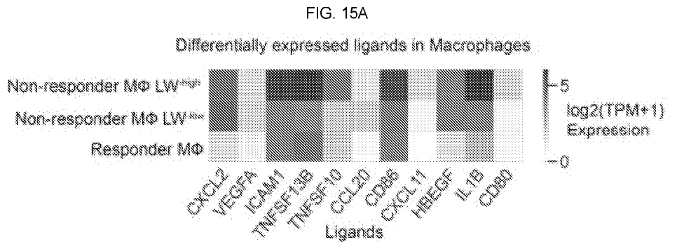

High LGALS1 and WARS-expressing macrophages are immuno-suppressive. Our analysis of sc-RNA-seq from melanoma and basal cell carcinoma and bulk RNA-seq from melanoma suggests that the expression of LGALS1 and WARS correlates with non-responsiveness to immunotherapy (FIGS. 12A, 12B) and poor overall survival (FIGS. 13A-13c). To determine whether high LGALS1 and WARS expressing macrophages (MΦ^{LW}-high) played a role in immunotherapy resistance, the biological processes and pathways enriched in macrophages were explored from non-responders compared to those from responders using Gene Set Enrichment Analysis (GSEA). Pathways enriched in MΦ^{LW}-high population, but not in MΦ^{LW}-low population of non-responders were then identified. The data showed that MΦ^{LW}-high population exhibited a significant overlap in the pathways related to the activation and polarization of macrophages and the interaction between macrophages and CD8 T cells (NES 2, FDR<0.05, FIG. 14A). For example, enrichment in interferon-gamma (IFN-γ) signaling and response was noted, which may indicate the polarization towards pro-inflammatory type macrophages (Muller et al., 2018), and upregulation of major histocompatibility complex class I (MHC-I), which could result in cytotoxic CD8 T cell activation and proliferation by processing and presenting the antigen to naïve T cells (Castro et al., 2018). Additionally, enrichment of the pattern recognition receptor signaling pathways was found, which include signaling receptors in TLR (toll-like receptor) and NLR (node-like receptor) families (FIG. 14A), indicating activation and differentiation of macrophages (Cen et al., 2018; Franchi et al., 2009; Singh et al., 2014). Together, these enriched pathways suggest that MΦ^{LW}-high in non-responders are fully activated and polarized. Thus, it is proposed that these mature macrophages could infiltrate into the TME as TAMs and induce differentiation and proliferation of naïve CD8 T cells to cytotoxic CD8 T cells via MHC-I antigen presentation. Further, these data suggest that CD8 T cells in non-responders could have already been activated by macrophages and exhausted prior anti-PD1 and anti-CTLA4 therapy. Therefore, it is posited that while TAMs themselves contribute to an immunosuppressive environment, their impact on the continuous activation of CD8 T cells could partially explain the resistance to immune checkpoint therapy.

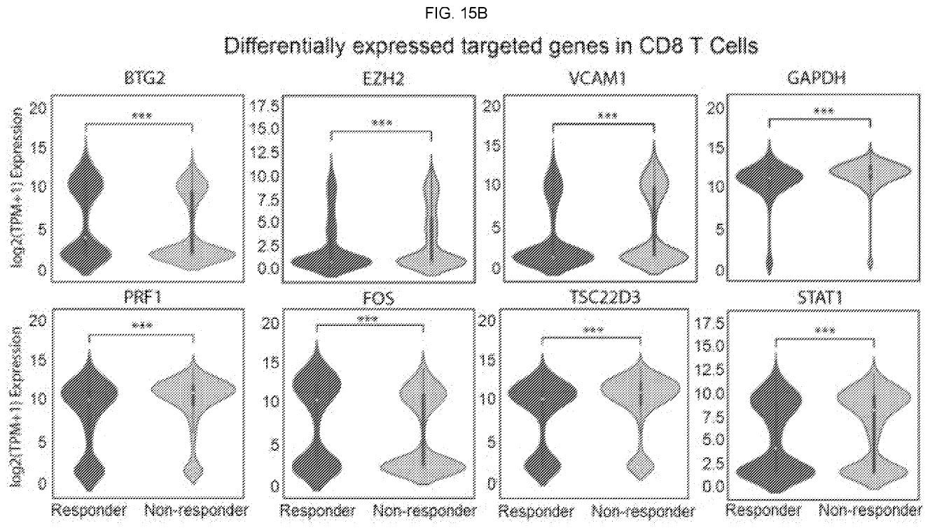

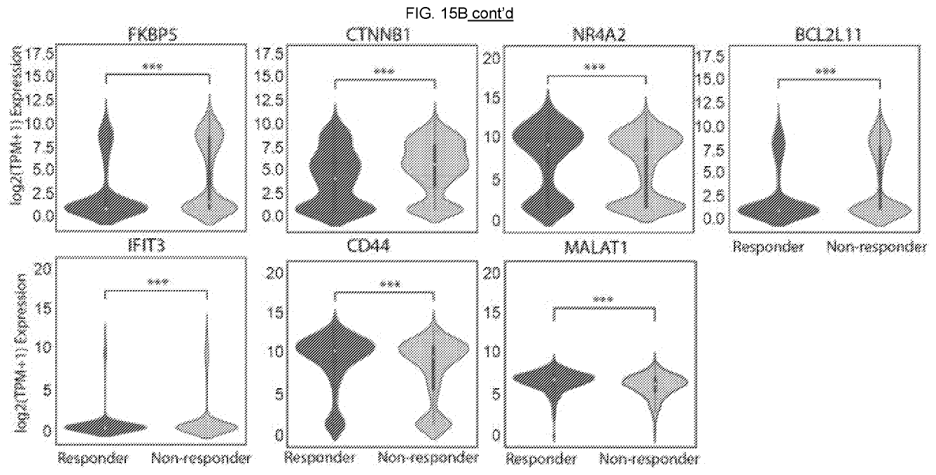

MΦ^{LW}-high populations from non-responders produce a set of ligands affecting CD8 T cells. It is hypothesized that the MΦ^{LW}-high population is immunosuppressive and may directly inhibit the function of CD8 T cells. Specifically, ligands or secreted factors from macrophages could contribute to the difference in the function and amount of CD8 T cells between responders and non-responders. To verify this, NichNet (Browaeys et al., 2020) was applied, a method that identifies ligands secreted by sender cells that could contribute to the differential gene expression in the receiver cells. First, all immune cells were designated as sender cells and CD8 T cells as receiver cells to identify ligands expressed in other immune cells that could affect CD8 T cell function between responders and non-responders. To exclude the impact of other immune cells from the result, a list of ligands was identified that are uniquely or dominantly expressed by macrophages (FIG. 14B). To examine the possible relationship between immunotherapy response and expression of LGALS1 and WARS in macrophages, the macrophages were separated from non-responders into two subpopulations as defined previously: MΦ^{LW}-high and MΦ^{LW}-low. Next, the Mann Whitney U test was applied to identify a subset of ligands differentially expressed between MΦ^{LW}-high and macrophages from responders. Among these ligands, CD80, CD86, TNFSF10 (TRAIL), TNFSF13B (TACI), and ICAM1 were found to be upregulated in MΦ^{LW}-high, while CXCL2, VEGFA, CCL20, CXCL11, HBGEF, and IL1B were overexpressed in both MΦ^{LW}high and MΦ^{LW}low compared to responders' macrophages (FIGS. 14B, 15). Next, macrophage-specific target genes in CD8 T cells affected by the ligands were determined (FIG. 14C). It was found that the CD8 T cells from non-responders had higher expression of GAPDH, EZH2, VCAM1, PRF1, TSCCD3 (GILZ), STAT1, FKBP5, IFIT3, CTNNB1, and BCL2L11, while CD8 T cells from responders expressed higher levels of BTG2, CD44, FOS, MALAT1 and NR4A2 (FIG. 14C). Gratifyingly, many of the genes identified as overexpressed in CD8 T cells from non-responders have known roles in T cell exhaustion and impaired function (Dimeloe et al., 2017;Kornberg et al., 2018; Levine et al., 2021), T cell differentiation (Kakaradov et al., 2017; Stairiker et al., 2020), and enhanced infiltration and recruitment of immune cells, including M2-macrophages (Pan et al., 2020; Xia et al., 2019). Together, these data suggest that secreted factors from MΦ^{LW}-high macrophages promote an over-activated or exhausted state in T-cells. Further studies are warranted to determine the mechanistic understanding of how ligands from MΦ^{LW}-high activate markers of T cell exhaustion.

Discussion. Although artificial intelligence (AI)-based approaches like neural networks represent a promising opportunity for identifying disease biomarkers, the lack of interpretability of these approaches impedes their application in biological sciences. Commonly referred to as ‘black-box’ models, it is increasingly difficult to comprehend the underlying biological mechanisms through which neural networks reach their predictions as their complexity increases. Due to this shortcoming, it is often more useful to employ simpler interpretable models irrespective of lower predictive accuracy over more complex models. In this example, an integrated approach was developed by combining neural networks and recursive feature elimination that allows accurate prediction of outcome and reveals the underlying features important for the predictions.

Neural networks disclosed herein were applied to sc-RNA-seq data from melanoma patients and identified a set of six genes, GZMB, GZMH, SELL, CCR7, LAGLS1, and WARS, that could predict a patient's response to ICB therapy. This finding was validated on a sc-RNA-seq dataset from basal cell carcinoma (Yost et al., 2019). Among the six genes, the biological impact of LGALS1 and WARS in macrophages were further investigated on other cell types in the microenvironment and the effectiveness of immunotherapy. GSEA of high LGALS1 and WARS-expressing macrophages indicated a heightened activation and polarization of the macrophage population. NicheNet was then applied to examine the impact of macrophages with high expression of LGALS1 and WARS on CD8 T cells. Ligands were found that mainly were or were uniquely secreted by macrophages, such as VEGFA, ICAM1, PLXNB2, targeted genes in CD8 T cells, and modulated activation, differentiation, and infiltration of naïve T cells. Further, the analyses of MΦ^{LW}-high/CD8 T cells revealed differentially expressed genes in CD8 T cells. For example, higher expression of CD44, EZH2, and BTG2 was found, which are known to suppress T cell function in CD8 T cells from patients with MΦ^{LW}-high macrophages. CD8 T cells from patients with high expression of LGALS1 and WARS seemed to be fully activated and differentiated into effector T cells. In contrast, the CD8 T cells from the responders of ICB therapy or patients with low expression of LGALS1 and WARS population overrepresented markers of quiescent T cell population and memory T cells. Since immune checkpoint therapy such as anti-PD1 and anti-CTLA4 aims to boost the immune system's potency and activate quiescent T cells, its effect could be reduced or diminished on already activated and exhausted T cells found in non-responders. Thus, the MΦ^{LW}-high macrophages-driven shift in T cell state could partially explain the differential response to ICB therapy.

The clinical response to ICB therapy is an elaborate consequence combining the interplay of several complex and multifaceted molecular mechanisms and signaling pathways in the TME, within and between cells. Current ICB therapy response prediction methods sacrifice the required complexity to develop computational models that can be interpreted. Compared to existing methods, disclosed neural networks can simultaneously model highly complex relations in data-driven by neural networks (known for their ability to model complex data) to predict patient outcomes and produce a set of descriptive genes that characterize non-responders and responders. The recursive gene elimination algorithm improves neural network prediction while concurrently reducing the number of genes into a set of smaller gene signatures. Consequently, these smaller gene signatures (<10) can easily be measured in clinical or pre-clinical settings to predict response to ICB therapy. Additionally, the ease of sc-RNA data collection allows for the rapid and straightforward collection of data required for accurate prediction of response.

Overall, a broadly-applicable, neural network-based approach is presented that uses recursive feature elimination and significance scoring to reduce a complex dataset (26,000 genes) into a clinically actionable biomarker gene set (6 genes) with 100% accuracy. It is also demonstrated that DeepGeneX accurately models complex biological data and elucidates the underlying molecular mechanisms behind the predictions. Furthermore, DeepGeneX is a generally applicable approach that can predict the effects from any dataset and in any disease context, given a training set of measurements. In conclusion, DeepGeneX is a significant step towards a more robust machine-based strategy for predicting phenotypic and clinical response to therapeutics with a complex mechanism of action, and as such, an essential addition to the current set of methodologies in this area.

Methods. Single-cell (sc) RNA Sequencing Data Analysis. The sc-RNA sequencing data and the corresponding patients' immunotherapy response and treatment record were achieved from the published paper (Sade-Feldman et al., 2018). The gene expression values of single cells were normalized as log2(TPM+1). Then, Seurat was applied to plot the immune cells of pre-treatment samples based on the normalized values of gene expression for each cell (Butler et al., 2018). The cell types were labeled according to the marker genes from the paper (Sade-Feldman et al., 2018). UMAPs from Seurat were plotted to show the different distribution of immune cell populations of responders and non-responders and show the differential expression of identified marker genes for predicting immune response. The Mann Whitney U test was applied to examine the statistical difference in expression of marker genes between responders and non-responders. Fisher Exact test was used to correlate the expression of two genes, where the threshold of high or low expression was defined as 2 of log2(TPM+1) value (Sade-Feldman et al., 2018). A dataset for basal cell carcinoma was also obtained and the data was processed with the above workflow to validate and generalize the findings (Yost et al., 2019).

Baseline Model Construction. Ppredictive models were constructed using gene expression values as inputs and immunotherapy responses as labels. Using the expression values of individual cells would result in overwhelming zero values. Therefore, the mean values of the expression values were used for genes within specific cell populations for each patient. Here, three conditions were considered: (1) all immune cells, (2) only CD8 T cells, and (3) only macrophages. Two baseline models were selected for the prediction of immunotherapy response: support vector machine (SVM) (M. A. Hearst, 1998) and XGBoost (Chen, 2016). SVM is a supervised predictive model that is used to classify the classes of points (vectors of numeric values, vectors of gene expression values per patients here specifically) by finding a hyperplane to separate these points in space. XGBoost, as a decision-tree based algorithm, works differently from SVM. Instead of identifying a plane, decision-tree like models construct a tree-like model that separates samples with each branching. More than a traditional decision tree model, XGB is able to adjust the existing tree models using the new input (gene expression data of patients and their response to immunotherapy) and minimize the prediction error via gradient boosting. For SVM, to avoid overfitting due to the unbalance of the sample space (19 patients) and the number of features (25,000+genes), genes with low variance (<1) were first excluded and sklearn build-in recursive feature elimination was applied to select the top 100 genes as final input features for model building (estimator as SVM, linear kernel) (Pedregosa et al., 2011). The performance of SVM (sigmoid kernel) using increasing percentages of the top 100 genes was evaluated by the average accuracy of 5-fold cross-validation. For XGBoost, due to its bagging property, all gene expression values were used as input features for self-selecting the features of importance. The performance of XGBoost was evaluated via a mean ROC curve with 5-fold cross-validation. The hyperparameters of XGBoost were tuned via a certain 70% of the dataset for optimal results and then fixed (learning rate=0.006, number of estimators=300, maximum tree depth=9).

Neural network Construction. Neural networks were developed using gene expression values as inputs and immunotherapy responses as output. As was done with XGBoost and SVM models, the mean expression values for genes for each patient were used, which eliminated hundreds of genes with 0 values. The implementation of the neural network was achieved using the Keras and TensorFlow Deep Learning libraries as described previously (Chan et al., 2021; Vijay and Gujral, 2020). A multi-phase Grid Search method was used to optimize the DNN hyperparameters (epochs, batch size, optimizer, weight initializer, hidden layer quantity, and nodes per hidden layer). Grid Search evaluates several hundred hyperparameter combinations in order to identify the model hyperparameters that result in the lowest binary cross-entropy error between observed and predicted responses to the drug. The error function that was used to compare numerous models was LOOCV (Leave-One-Out-Cross-Validation) Binary Cross-Entropy, as it is a commonly used error function for binary classification. In LOOCV, each time n−1 patients' scRNA data and response are used to train the model to predict the remaining patient's response. The process is repeated n times, excluding and predicting every patient. Binary cross entropy between predicted and observed responses is used to assign an error score to each model built with various combinations of hyperparameter values. In each phase of Grid Search, combinations of hyperparameters were evaluated, and the combination with the lowest LOOCV Binary cross-entropy was used in the subsequent phase of optimization until the final phase was reached. After the optimal hyperparameters were identified, the architecture was used to build the preliminary DeepGeneX network.

Recursive Gene Elimination. After the development of a trained preliminary neural network defined by f and a dataset defined by [y, X₁, X₂, X₃, . . . ], the baseline error (e_{baseline}) can be computed, assuming a pre-defined cost function. The cost function was defined as follows:

$$C ( Binary Cross Entropy ) = - \frac{1}{n} {\sum }_{i = 1}^{n} {y}_{i} * log {\hat{y}}_{i} + ( 1 - {y}_{i} ) * log ( 1 - {\hat{y}}_{i} ) \qquad Equation 9$$

The baseline error was calculated as defined below:

$${e}_{baseline} = \frac{1}{n} {\sum }_{i = 1}^{n} C ( f ( {y}_{i} , {X}_{1} , {X}_{2} , {X}_{3} , … ) ) \qquad Equation 10$$

To calculate the post-permutation error (eₚₑᵣₘᵤₜₐₜᵢₒₙ), each feature is shuffled one-by-one for a total of 200 random shuffles. The matrix of features with a single feature permuted once can defined by X^{permutation}. Accordingly, the post-permutation error for an individual feature is computed as follows:

$${e}_{permutation} = \frac{1}{200} {\sum }_{j = 1}^{200} \frac{1}{n} {\sum }_{i = 1}^{n} C ( f ( y , {X}_{i}^{permutation} ) ) \qquad Equation 11$$

Lastly, the relative gene importance (RGI) or error difference for an individual feature is computed:

$$RGI = {e}_{permutation} - {e}_{baseline} \qquad Equation 12$$

For each gene, an RGI score was computed, and the genes are ranked from highest to lowest importance. In the first round of elimination, the top 1000 genes based on RGI were selected to build the new model. Subsequently, for every following round of recursive gene elimination, the bottom 25% of genes were eliminated. Using just the top 75% of genes, a new DeepGeneX model was built, and LOOCV accuracy was used to track the model's overall relative performance across several rounds. This three-step process—(1) ranking genes by importance score, (2) removing the bottom 25% of genes, and (3) assessing LOOCV accuracy of the DeepGeneX model built using only the remaining genes is repeated until the LOOCV Accuracy of the model achieves an inflection point where the accuracy starts to decrease as the number of inputs decrease.

Cell Interaction Analysis. NicheNet was adopted to examine the difference in cell-cell interaction in the tumor microenvironment between responders and non-responders, especially from the aspect of how macrophages would affect CD8 T cells (Browaeys et al., 2020). By specifying the cell types of sender and receiver cells and the condition to compare with, NicheNet identified ligands of the sender cells that were likely to cause the differential gene expression in the receiver cells between two conditions: responder to immunotherapy or not in this case. Here, CD8 T cells were first chosen as receivers and macrophages as senders to obtain ligands produced by macrophages that could contribute to the difference in CD8 T cells between responders and non-responders. The situation was also examined where all immune cells were considered as senders and CD8 T cells as receivers. The overlapping ligands from these two analyses identified the ligands that were uniquely or mostly secreted by macrophages that would not be masked by background—the interaction between CD8 T cell and other immune cells. It also provided the information of the possible targeted genes in CD8 T cells of these identified ligands, allowing the linking of this information and the following-up pathway enrichment analysis. To examine how LGALS1 and WARS expression in macrophages would contribute to the resistance to immunotherapy, MΦ^{LW}high were considered as macrophages with high expression of both LGALS1 and WARS, while the rest as MΦ^{LW}-low, using the expression threshold defined above. The expression of identified ligands were compared from the MΦ^{LW}-high/MΦ^{LW}-low population of non-responders to those from responders. Mann Whitney U test was applied to show the significant difference in ligand expression.

GSEA Analysis. GSEA analysis on the gene expression data of specific immune cell populations to investigate the distinction in pathway regulation between patients with different immune responses or marker gene expressions (Subramanian et al., 2005), using the GO biological process pathway dataset. Differentially regulated pathways were focused on that are enriched macrophages from non-responder compared to those from responders. Pathways with a false discovery rate less than 0.05 and a normalized enrichment score of more than two were kept. To identify pathway enrichments that are dominantly impacted by MΦ^{LW}-high, the macrophages were separated from non-responders by their LGALS1 and WARS expression and compared the enriched pathways compared to macrophages from responders accordingly. The pathways enriched in non-responders were then intersected with those upregulated in MΦ^{LW}-high, but not in MΦ^{LW}-low to achieve a final list of pathways that are uniquely enriched in MΦLW-high from non-responders and could contribute to the distinct immunotherapy response.

Survival Analysis. The clinical data (overall survival data) and the expression data (htseq-count) of seventeen cancer types were achieved from the TCGA database, GDC portal (Grossman et al., 2016). The expression data were normalized to CPM (counts per million) value using edgeR (Robinson et al., 2010). To examine the impact of the expression of marker genes (SELL, CCR7, WARS and LGALS1) on patients' survival, for each cancer types, patients were split into two groups: one with favorable gene expression pattern and the other with the unfavorable pattern. To determine the expression pattern, the expression values for four marker genes were first ranked across patients, SELL/CCR7 in descending order, since DeepGeneX indicates that the higher expression of these two genes linked with immunotherapy response, while LGALS1/WARS in ascending order. The rank value of these four genes were then summed for each patient. For each cancer type, if the sum value of a patient is greater than the median, that patient is classified to have an unfavorable expression pattern, otherwise, favorable. Kaplan-Meier analysis was used to compare the survival difference between these two groups of patients and generate corresponding survival curve. Log rank test was used to examine the statistical significance of such difference.

### REFERENCES FOR EXPERIMENTAL EXAMPLE 2 & ASSOCIATED FIGURE DESCRIPTIONS

Abebayehu, D., Spence, A., Boyan, B.D., Schwartz, Z., Ryan, J.J., and McClure, M.J. (2017). Galectin-1 promotes an M2 macrophage response to polydioxanone scaffolds. J Biomed Mater Res A 105, 2562-2571.
Bai, R., Lv, Z., Xu, D., and Cui, J. (2020). Predictive biomarkers for cancer immunotherapy with immune checkpoint inhibitors. Biomarker Research 8, 1-17.
Binnewies, M., Roberts, E.W., Kersten, K., Chan, V., Fearon, D.F., Merad, M., Coussens, L.M., Gabrilovich, D.I., Ostrand-Rosenberg, S., and Hedrick, C.C. (2018). Understanding the tumor immune microenvironment (TIME) for effective therapy. Nature medicine 24, 541-550.
Browaeys, R., Saelens, W., and Saeys, Y. (2020). NicheNet: modeling intercellular communication by linking ligands to target genes. Nat Methods 17, 159-162.
Brown, J., Wallet, M.A., Krastins, B., Sarracino, D., and Goodenow, M.M. (2010). Proteome bioprofiles distinguish between M1 priming and activation states in human macrophages. J Leukoc Biol 87, 655-662.
Butler, A., Hoffman, P., Smibert, P., Papalexi, E., and Satija, R. (2018). Integrating single-cell transcriptomic data across different conditions, technologies, and species. Nat Biotechnol 36, 411-420.
Castro, F., Cardoso, A.P., Goncalves, R.M., Serre, K., and Oliveira, M.J. (2018). Interferon-Gamma at the Crossroads of Tumor Immune Surveillance or Evasion. Front Immunol 9, 847.
Cen, X., Liu, S., and Cheng, K. (2018). The Role of Toll-Like Receptor in Inflammation and Tumor Immunity. Front Pharmacol 9, 878.
Chan, M., Vijay, S., McNevin, J., McElrath, M.J., Holland, E.C., and Gujral, T.S. (2021). Machine learning identifies molecular regulators and therapeutics for targeting SARS-CoV2-induced cytokine release. Molecular systems biology 17, e10426.
Chen, Q., Han, B., Meng, X., Duan, C., Yang, C., Wu, Z., Magafurov, D., Zhao, S., Safin, S., Jiang, C., et al. (2019). Immunogenomic analysis reveals LGALS1 contributes to the immune heterogeneity and immunosuppression in glioma. Int J Cancer 145, 517-530.
Chen, T.a.G., Carlos (2016). XGBoost: A Scalable Tree Boosting System (San Francisco, California, USA: ACM).
Dimeloe, S., Burgener, A.V., Grahlert, J., and Hess, C. (2017). T-cell metabolism governing activation, proliferation and differentiation; a modular view. Immunology 150, 35-44.
Dwary, A.D., Master, S., Patel, A., Cole, C., Mansour, R., Mills, G., Koshy, N., Peddi, P., Burton, G., Hammoud, D., et al. (2017). Excellent response to chemotherapy post immunotherapy. Oncotarget 8, 91795-91802.
Esfahani, K., Roudaia, L., Buhlaiga, N., Del Rincon, S.V., Papneja, N., and Miller, W.H., Jr. (2020). A review of cancer immunotherapy: from the past, to the present, to the future. Curr Oncol 27, S87-S97.
Franchi, L., Warner, N., Viani, K., and Nunez, G. (2009). Function of Nod-like receptors in microbial recognition and host defense. Immunol Rev 227, 106-128.
Grossman, R.L., Heath, A.P., Ferretti, V., Varmus, H.E., Lowy, D.R., Kibbe, W.A., and Staudt, L.M. (2016). Toward a Shared Vision for Cancer Genomic Data. N Engl J Med 375, 1109-1112.
Hashimoto, K., Kouno, T., Ikawa, T., Hayatsu, N., Miyajima, Y., Yabukami, H., Terooatea, T., Sasaki, T., Suzuki, T., Valentine, M., et al. (2019). Single-cell transcriptomics reveals expansion of cytotoxic CD4 T cells in supercentenarians. Proc Natl Acad Sci U S A 116, 24242-24251.
Kakaradov, B., Arsenio, J., Widjaja, C.E., He, Z., Aigner, S., Metz, P.J., Yu, B., Wehrens, E.J., Lopez, J., Kim, S.H., et al. (2017). Early transcriptional and epigenetic regulation of CD8(+) T cell differentiation revealed by single-cell RNA sequencing. Nat Immunol 18, 422-432
Kornberg, M.D., Bhargava, P., Kim, P.M., Putluri, V., Snowman, A.M., Putluri, N., Calabresi, P.A., and Snyder, S.H. (2018). Dimethyl fumarate targets GAPDH and aerobic glycolysis to modulate immunity. Science 360, 449-453.
Lapuente-Santana, Ó., van Genderen, M., Hilbers, P.A., Finotello, F., and Eduati, F. (2021). Interpretable systems biomarkers predict response to immune-checkpoint inhibitors. Patterns 2, 100293.
Levine, L.S., Hiam-Galvez, K.J., Marquez, D.M., Tenvooren, I., Madden, M.Z., Contreras, D.C., Dahunsi, D.O., Irish, J.M., Oluwole, O.O., Rathmell, J.C., et al. (2021). Single-cell analysis by mass cytometry reveals metabolic states of early-activated CD8(+) T cells during the primary immune response. Immunity 54, 829-844 e825.
M. A. Hearst, S.T.D., E. Osuna, J. Platt and B. Scholkopf (1998). Support vector machines. IEEE Intelligent Systems and their Applications 13, 18-28.
Martin, M.D., and Badovinac, V.P. (2018). Defining Memory CD8 T Cell. Front Immunol 9, 2692.
Muller, E., Speth, M., Christopoulos, P.F., Lunde, A., Avdagic, A., Oynebraten, I., and Corthay, A. (2018). Both Type I and Type II Interferons Can Activate Antitumor M1 Macrophages When Combined With TLR Stimulation. Front Immunol 9, 2520.
Nie, A., Sun, B., Fu, Z., and Yu, D. (2019). Roles of aminoacyl-tRNA synthetases in immune regulation and immune diseases. Cell Death Dis 10, 901.
Pan, H., Wang, X., Huang, W., Dai, Y., Yang, M., Liang, H., Wu, X., Zhang, L., Huang, W., Yuan, L., et al. (2020). Interferon-Induced Protein 44 Correlated With Immune Infiltration Serves as a Potential Prognostic Indicator in Head and Neck Squamous Cell Carcinoma. Front Oncol 10, 557157.
Pedregosa, F.a.V., G. and Gramfort, A. and Michel, V., and Thirion, B.a.G., O. and Blondel, M. and Prettenhofer, P., and Weiss, R.a.D., V. and Vanderplas, J. and Passos, A. and, and Cournapeau, D.a.B., M. and Perrot, M. and Duchesnay, E. (2011). Scikit-learn: Machine Learning in Python. Journal of Machine Learning Research 12, 2825-2830.
Robinson, M.D., Mccarthy, D.J., and Smyth, G.K. (2010). edgeR: a Bioconductor package for differential expression analysis of digital gene expression data. Bioinformatics 26, 139-140.
Sade-Feldman, M., Yizhak, K., Bjorgaard, S.L., Ray, J.P., de Boer, C.G., Jenkins, R.W., Lieb, D.J., Chen, J.H., Frederick, D.T., Barzily-Rokni, M., et al. (2018). Defining T Cell States Associated with Response to Checkpoint Immunotherapy in Melanoma. Cell 175, 998-1013 e1020.
Singh, M., Khong, H., Dai, Z., Huang, X.F., Wargo, J.A., Cooper, Z.A., Vasilakos, J.P., Hwu, P., and Overwijk, W.W. (2014). Effective innate and adaptive antimelanoma immunity through localized TLR7/8 activation. J Immunol 193, 4722-4731.
Staff, N. (2019). New Drugs, New Side Effects: Complications of Cancer Immunotherapy was originally published by the National Cancer Institute.
Stairiker, C.J., Thomas, G.D., and Salek-Ardakani, S. (2020). EZH2 as a Regulator of CD8+ T Cell Fate and Function. Front Immunol 11, 593203.
Subramanian, A., Tamayo, P., Mootha, V.K., Mukherjee, S., Ebert, B.L., Gillette, M.A., Paulovich, A., Pomeroy, S.L., Golub, T.R., Lander, E.S., et al. (2005). Gene set enrichment analysis: a knowledge-based approach for interpreting genome-wide expression profiles. Proc Natl Acad Sci U S A 102, 15545-15550.
Ventola, C.L. (2017). Cancer Immunotherapy, Part 3: Challenges and Future Trends. P T 42, 514-521.
Vera Aguilera, J., Paludo, J., McWilliams, R.R., Zhang, H., Li, Y., Kumar, A.B., Failing, J., Kottschade, L.A., Block, M.S., Markovic, S.N., et al. (2020). Chemo-immunotherapy combination after PD-1 inhibitor failure improves clinical outcomes in metastatic melanoma patients. Melanoma Res 30, 364-375.
Vijay, S., and Gujral, T.S. (2020). Non-linear Deep Neural Network for Rapid and Accurate Prediction of Phenotypic Responses to Kinase Inhibitors. Iscience 23, 101129.
Waldman, A.D., Fritz, J.M., and Lenardo, M.J. (2020). A guide to cancer immunotherapy: from T cell basic science to clinical practice. Nat Rev Immunol 20, 651-668.
Xia, A., Zhang, Y., Xu, J., Yin, T., and Lu, X.J. (2019). T Cell Dysfunction in Cancer Immunity and Immunotherapy. Front Immunol 10, 1719.
Yost, K.E., Satpathy, A.T., Wells, D.K., Qi, Y., Wang, C., Kageyama, R., McNamara, K.L., Granja, J.M., Sarin, K.Y., Brown, R.A., et al. (2019). Clonal replacement of tumor-specific T cells following PD-1 blockade. Nat Med 25, 1251-1259.
Zupan, J. Introduction to artificial neural network (ANN) methods: what they are and how to use them. Acta Chimica Slovenica 41, 327-327 (1994).
Grapov, D., Fahrmann, J., Wanichthanarak, K. and Khoomrung, S. (2018). Rise of Deep Learning for Genomic, Proteomic, and Metabolomic Data Integration in Precision Medicine.

Closing Paragraphs. Unless otherwise indicated, the practice of the present disclosure can employ conventional techniques of immunology, molecular biology, microbiology, cell biology and recombinant DNA. These methods are described in the following publications. See, e.g., Sambrook, et al. Molecular Cloning: A Laboratory Manual, 2nd Edition (1989); F. M. Ausubel, et al. eds., Current Protocols in Molecular Biology, (1987); the series Methods IN Enzymology (Academic Press, Inc.); M. MacPherson, et al., PCR: A Practical Approach, IRL Press at Oxford University Press (1991); MacPherson et al., eds. PCR 2: Practical Approach, (1995); Harlow and Lane, eds. Antibodies, A Laboratory Manual, (1988); and R. I. Freshney, ed. Animal Cell Culture (1987).

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Eds. Attwood T et al., Oxford University Press, Oxford, 2006).

What is claimed is:

1. A method comprising:
receiving a training data set comprising a set of biological input features and an observed response of a biological system to the set of biological input features or a treatment or therapy applied to the biological system;
training, based at least in part on the training data set, a model to predict a response of the biological system to the set of biological input features;
determining a baseline error associated with the model based at least in part on a first difference between a predicted response determined by the model and the observed response of the biological system;
determining permuted input data by altering a first feature of the set of biological input features;
determining, by the model and based at least in part on the permuted input data, an updated prediction of the biological system to the permuted input data;
determining a permutation error associated with the updated prediction based at least in part on a second difference between the updated prediction and the observed response;
determining an importance score associated with the first feature based at least in part on a difference between the baseline error and the permutation error;
determining, based at least in part on the importance score and one or more importance score associated with one or more other input features of the set of biological input features, a ranking of the first input feature within the set of biological input features; and
determining, based at least in part on the ranking, a subset of biological input features from the set of biological input features.

2. The method of claim 1, wherein:
the set of biological input features is a first set associated with a first biological sample;
the method further comprises receiving a second set of biological input features associated with a second biological sample; and
determining the permuted input data comprises exchanging a first portion of the first set with a second portion of the second set.

3. The method of either claim 1 or 2, wherein the model is a neural network and training the model comprises:
determining, by the model and based at least in part on the set of biological input features, an estimated response of the biological system;
determining a loss based at least in part on a third difference between the estimated response and the observed response of the biological system; and
altering one or more parameters of a component of the model to reduce the loss.

4. The method of claim 3, wherein training the model further includes determining one or more hyperparameters associated with the model, the hyperparameters including at least one of:
an epoch;
a batch size;
an optimizer algorithm type;
an initial weighting scheme;
a hidden layer quantity;
a number of nodes per hidden layer;
a number of layers containing one or more dropouts;
a percentage of dropouts in a layer that includes dropouts; or
an activation function type.

5. The method of any one of claims 1-4, wherein the first input feature includes an activity profile associated with at least one of:
a small molecule;
a kinase inhibitor;
a protein;
a biomarker;
an RNA sequence; and
a DNA sequence.

6. The method of claim 5, wherein the activity profile quantifies an extent to which an input feature acts upon a molecular pathway or molecular mechanism or a extent to which the input feature influences a response of the biological system to a treatment or therapy.

7. The method of any one of claims 1-6, wherein the first input feature includes at least one of:
a small molecule;
a kinase inhibitor;
a protein;
a biomarker;
an RNA sequence; and
a DNA sequence.

8. The method of any one of claims 1-7, wherein determining the subset of biological input features from the set of biological input features comprises determining that the subset of biological input features is associated with a top r percentage of the set of biological input features by importance score ranking.

9. The method of any one of claims 1-8, wherein the observed response of the biological system comprises one of:
a quantified viability of a cell;
an immune checkpoint therapy response; or
an immune response to inoculation.

10. The method of any one of claims 1-9, wherein the set of biological input features comprises different kinases, kinase inhibitors, or activity profiles associated with different kinases or kinase inhibitors.

11. The method of any one of claims 1-10, wherein the observed response of the biological system includes a viability of at least one of an epithelial or mesenchymal cancer cell or a response to ICB therapy.

12. The method of any one of claims 1-11, wherein the model is a neural network and training the model comprises:
determining, by the model and based at least in part on the set of biological input features, an estimated response of the biological system;
determining a loss based at least in part on a third difference between the estimated response and the observed response of the biological system; and
altering one or more parameters of a component of the model to reduce the loss.

13. A system comprising:
one or more processors; and
a memory storing processor-executable instructions that, when executed by the one or more processors, cause the system to perform operations comprising:
receiving a training data set comprising a set of biological input features and an observed response of a biological system associated with the set of biological input features;
training, based at least in part on the training data set, a model to predict a response of the biological system to the set of biological input features;
determining a baseline error associated with the model based at least in part on a first difference between a predicted response determined by the model and the observed response of the biological system;
determining permuted input data by altering a first feature of the set of biological input features;
determining, by the model and based at least in part on the permuted input data, an updated prediction of the biological system to the permuted input data;
determining a permutation error associated with the updated prediction based at least in part on a second difference between the updated prediction and the observed response;
determining an importance score associated with the first feature based at least in part on a difference between the baseline error and the permutation error;
determining, based at least in part on the importance score and one or more importance score associated with one or more other input features of the set of biological input features, a ranking of the first input feature within the set of biological input features; and
determining, based at least in part on the ranking, a subset of biological input features from the set of biological input features.

14. The system of claim 13, wherein the model is a neural network and training the model comprises:
determining, by the model and based at least in part on the set of biological input features, an estimated response of the biological system;
determining a loss based at least in part on a third difference between the estimated response and the observed response of the biological system; and
altering one or more parameters of a component of the model to reduce the loss.

15. The system of either claim 13 or 14, wherein training the model further includes determining one or more hyperparameters associated with the model, the hyperparameters including at least one of:
an epoch;
a batch size;
an optimizer algorithm type;
an initial weighting scheme;
a hidden layer quantity;
a number of nodes per hidden layer;
a number of layers containing one or more dropouts;
a percentage of dropouts in a layer that includes dropouts; or
an activation function type.

16. The system of any one of claims 13-15, wherein the first input feature includes an activity profile associated with at least one of:
a small molecule;
a kinase inhibitor;
a protein;
a biomarker;
an RNA sequence; and
a DNA sequence.

17. The system of claim 16, wherein the activity profile quantifies an extent to which an input feature acts upon a molecular pathway or molecular mechanism.

18. The system of any one of claims 13-17, wherein the first input feature includes at least one of:
a small molecule;
a kinase inhibitor;
a protein;
a biomarker;
an RNA sequence; and
a DNA sequence.

19. The system of any one of claims 13-18, wherein determining the subset of biological input features from the set of biological input features comprises determining that the subset of biological input features is associated with a top r percentage of the set of biological input features by importance score ranking.

20. The system of any one of claims 13-19, wherein the observed response of the biological system comprises one of:
a quantified viability of a cell;
an immune checkpoint therapy response; or
an immune response to inoculation.

21. The system of any one of claims 13-20, wherein the set of biological input features comprises different kinases, kinase inhibitors, or activity profiles associated with different kinases or kinase inhibitors.

22. The system of any one of claims 13-21, wherein the observed response of the biological system includes a viability of at least one of an epithelial or mesenchymal cancer cell.

23. The system of any one of claims 13-22, wherein the set of biological input features comprises a plurality of RNA loci.

24. The system of any one of claims 13-23, wherein the observed response of the biological system includes a response to ICB therapy, a pharmaceutical treatment or administration, or a therapy applied to the biological system.

25. The system of any one of claims 13-24, wherein the model is a neural network and training the model comprises:
determining, by the model and based at least in part on the set of biological input features, an estimated response of the biological system;
determining a loss based at least in part on a third difference between the estimated response and the observed response of the biological system; and
altering one or more parameters of a component of the model to reduce the loss.

26. The system of any one of claims 13-25, wherein:
the set of biological input features is a first set associated with a first biological sample;
the operations further comprise receiving a second set of biological input features associated with a second biological sample; and
determining the permuted input data comprises exchanging a first portion of the first set with a second portion of the second set.

27. A non-transitory computer-readable medium comprising computer-executable instructions that, when executed by one or more processors, cause the one or more processors to perform operations comprising:
receiving a training data set comprising a set of biological input features and an observed response of a biological system to the set of biological input features;
training, based at least in part on the training data set, a model to predict a response of the biological system to the set of biological input features;
determining a baseline error associated with the model based at least in part on a first difference between a predicted response determined by the model and the observed response of the biological system;
determining permuted input data by altering a first feature of the set of biological input features;
determining, by the model and based at least in part on the permuted input data, an updated prediction of the biological system to the permuted input data;
determining a permutation error associated with the updated prediction based at least in part on a second difference between the updated prediction and the observed response;
determining an importance score associated with the first feature based at least in part on a difference between the baseline error and the permutation error;
determining, based at least in part on the importance score and one or more importance score associated with one or more other input features of the set of biological input features, a ranking of the first input feature within the set of biological input features; and
determining, based at least in part on the ranking, a subset of biological input features from the set of biological input features.

28. The non-transitory computer-readable medium of claim 27, wherein the model is a neural network and training the model comprises:
determining, by the model and based at least in part on the set of biological input features, an estimated response of the biological system;
determining a loss based at least in part on a third difference between the estimated response and the observed response of the biological system; and
altering one or more parameters of a component of the model to reduce the loss.

29. The non-transitory computer-readable medium of either claim 27 or 28, wherein training the model further includes determining one or more hyperparameters associated with the model, the hyperparameters including at least one of:
an epoch;
a batch size;
an optimizer algorithm type;
an initial weighting scheme;
a hidden layer quantity;
a number of nodes per hidden layer;
a number of layers containing one or more dropouts;
a percentage of dropouts in a layer that includes dropouts; or
an activation function type.

30. The non-transitory computer-readable medium of any one of claims 27-29, wherein the first input feature includes an activity profile associated with at least one of:
a small molecule;
a kinase inhibitor;
a protein;
a biomarker;
an RNA sequence; and
a DNA sequence.

31. The non-transitory computer-readable medium of claim 30, wherein the activity profile quantifies an extent to which an input feature acts upon a molecular pathway or molecular mechanism.

32. The non-transitory computer-readable medium of any one of claims 27-31, wherein the first input feature includes at least one of:
a small molecule;
a kinase inhibitor;
a protein;
a biomarker;
an RNA sequence; and
a DNA sequence.

33. The non-transitory computer-readable medium of any one of claims 27-32, wherein determining the subset of biological input features from the set of biological input features comprises determining that the subset of biological input features is associated with a top r percentage of the set of biological input features by importance score ranking.

34. The non-transitory computer-readable medium of any one of claims 27-33, wherein the observed response of the biological system comprises one of:
a quantified viability of a cell;
an immune checkpoint therapy response; or
an immune response to inoculation.

35. The non-transitory computer-readable medium of any one of claims 27-34, wherein the set of biological input features comprises different kinases, kinase inhibitors, or activity profiles associated with different kinases or kinase inhibitors.

36. The non-transitory computer-readable medium of any one of claims 27-35, wherein the observed response of the biological system includes a viability of at least one of an epithelial or mesenchymal cancer cell.

37. The non-transitory computer-readable medium of any one of claims 27-36, wherein the set of biological input features comprises a plurality of RNA loci.

38. The non-transitory computer-readable medium of any one of claims 27-37, wherein the observed response of the biological system includes a response to ICB therapy.

39. The non-transitory computer-readable medium of any one of claims 27-38, wherein the model is a neural network and training the model comprises:
determining, by the model and based at least in part on the set of biological input features, an estimated response of the biological system;
determining a loss based at least in part on a third difference between the estimated response and the observed response of the biological system; and
altering one or more parameters of a component of the model to reduce the loss.

40. The non-transitory computer-readable medium of any one of claims 27-39, wherein:
the set of biological input features is a first set associated with a first biological sample;
the operations further comprise receiving a second set of biological input features associated with a second biological sample; and
determining the permuted input data comprises exchanging a first portion of the first set with a second portion of the second set.
